(19) United States
(12) Patent Application Publication
(10) Pub. No.: US 2024/0092840 A1
(43) Pub. Date: Mar. 21, 2024

(54) VACCINE FORMULATION COMPRISING RECOMBINANT OVERLAPPING PEPTIDES AND NATIVE PROTEINS

(71) Applicant: OXFORD VACMEDIX UK LIMITED, Oxford (GB)

(72) Inventor: Shisong JIANG, Oxfordshire (GB)

(21) Appl. No.: 18/506,420

(22) Filed: Nov. 10, 2023

Related U.S. Application Data
(63) Continuation of application No. PCT/GB2022/051175, filed on May 9, 2022.

(30) Foreign Application Priority Data
May 11, 2021 (GB) 2106713.7

(51) Int. Cl. C07K 14/005; A61K 39/12; A61K 39/215; A61P 37/04; C07K 14/47

(52) U.S. Cl. CPC C07K 14/005; A61K 39/12; A61K 39/215; A61P 37/04; C07K 14/4747; C07K 2319/21; C07K 2319/50; C12N 2710/20022; C12N 2710/20034; C12N 2710/20071; C12N 2770/20022; C12N 2770/20034; C12N 2770/20071

(57) ABSTRACT

The invention provides formulations, compositions, and kits comprising polypeptides and native proteins or portions thereof for the immunization and/or treatment of a subject, or polypeptides encoding said polypeptides and native proteins or portions thereof, as well as methods of treatment using said formulations, compositions, and kits, and methods of manufacture of said formulations, compositions, and kits.

### CROSS-REFERENCE TO RELATED APPLICATIONS

This application is a continuation of International Patent Application No. PCT/GB2022/051175 filed May 9, 2022, which application claims the benefit of United Kingdom Patent Application No. 2106713.7 filed on May 11, 2021, both of which are incorporated herein by reference in their entireties.

### FIELD OF THE INVENTION

The invention provides formulations, compositions, and kits comprising polypeptides and native proteins or portions thereof for the immunization and/or treatment of a subject, or polypeptides encoding said polypeptides and native proteins or portions thereof, as well as methods of treatment using said formulations, compositions, and kits, and methods of manufacture of said formulations, compositions, and kits.

### BACKGROUND OF THE INVENTION

The efficacy of vaccines is, of course, critical in their ability to effectively mitigate diseases. Vaccines can be prophylactic—protecting against disease—or therapeutic—treating an existing disease. In the case of prophylactic vaccines, whilst it is elementary that a more efficacious vaccine is desirable, in some cases there is a minimum efficacy threshold for a vaccine to be effective in mitigating a disease. For example, in a recent study, it was shown that in order for a vaccine to effectively prevent the SARS-CoV-2 epidemic, the efficacy threshold for a vaccine as a sole intervention was that it must be at least 60% effective at preventing infection to reduce the peak of the number of infections by 99% (Bartsch et al., 2020). The same study showed that in order to extinguish an active epidemic where 5% of the population have been exposed to the virus, vaccine efficacy has to be at least 60% to reduce the peak by 85% assuming 100% coverage, rising to 80% when coverage drops to 75%, and 100% when coverage drops to 60%. It is clear, therefore, that boosting vaccine efficacy is not only desirable, but necessary, especially in the context of the early stage of an epidemic where coverage is unlikely to be high.

In the case of therapeutic vaccines, for example cancer immunotherapeutic vaccine technologies, it is acknowledged that the single most important factor is the choice of antigen (Hollingsworth and Jansen, 2019). However, other strategies have been attempted to boost the efficacy of such vaccines. For example, by combining them with checkpoint inhibitors, adjuvants, cytokines, chemotherapeutic agents, and others. Nonetheless, such strategies still have significant hurdles which need to be overcome in order to safely improve efficacy and progress to the clinic.

What is required is an approach which improves the efficacy of a given vaccine without introducing further significant technical hurdles or compromising patient safety. Surprisingly, applicants have found that introducing a recombinant overlapping peptide vaccine at the same time as a native protein sequence, or a portion or fragment thereof, leads to an increase in the magnitude of the immune response of the subject, thus indicating an increased efficacy of the vaccine regime. The applicants have found that this may have broad general applicability to vaccine combined therapeutic approaches.

### SUMMARY OF THE INVENTION

In one aspect, the invention provides a formulation for the immunization and/or treatment of a subject comprising a polypeptide comprising two or more peptide fragments, wherein a first peptide fragment comprises a first sequence derived from a native protein sequence and wherein a second peptide fragment comprises a second sequence derived from the native protein sequence, further comprising one or more protease cleavage site sequences located between each of the two or more peptide fragments; and the native protein sequence or a portion thereof.

In some embodiments, the two or more peptide fragments comprise one or more overlapping sequences. In some embodiments, the one or more overlapping sequences are between 2 and 31 amino acids in length, optionally wherein the one or more overlapping sequences are at least 8 amino acids in length. In some embodiments, the one or more protease cleavage site sequences is an exogenous protease cleavage site, optionally a cathepsin cleavage sequence, preferably cathepsin S, more preferably an LRMK cleavage sequence. In some embodiments, the polypeptide comprises three or more peptide fragments, preferably five or more peptide fragments, more preferably ten or more peptide fragments.

In some embodiments, the formulation further comprises a pharmaceutically acceptable carrier.

In some embodiments, the formulation further comprises an adjuvant, preferably Monophosphate Lipid A (MPL), montanide, alum-based adjuvants, oil-in-water, or water-in-oil, more preferably Monophosphate Lipid A, montanide, or alum-based adjuvants.

In some embodiments, the concentration of the polypeptide is between 10 to 10000 μg·kg⁻¹ and the concentration of the native protein sequence or portion thereof is between 10 to 10000 μg·kg⁻¹.

In some embodiments, the native protein sequence is the S protein of a coronavirus. In some embodiments, the coronavirus is a betacoronavirus, optionally a severe acute respiratory syndrome-related coronavirus, optionally SARS-CoV-2. In some embodiments, the coronavirus is a human coronavirus. In some embodiments, at least two of the two or more peptide fragments of the polypeptide comprise sequences derived from the S1 and/or S2 subunit of the S protein and/or wherein the portion of the native protein sequence comprises sequences derived from the S1 and/or S2 subunit of the S protein.

In some embodiments at least one of the two or more peptide fragments comprises a sequence derived from the receptor binding domain (RBD), optionally the receptor binding motif (RBM) of the S1 subunit and/or wherein the portion of the native protein sequence comprises the receptor binding domain (RBD), optionally the receptor binding motif (RBM) of the S1 subunit.

In some embodiments, at least one of the two or more peptide fragments comprises a sequence derived from the HR2 and/or HR1 domain of the S2 subunit and/or wherein the portion of the native protein sequence comprises the HR2 and/or HR1 domain of the S2 subunit.

In some embodiments the native protein sequence is survivin, chosen from any one of the following survivin isoforms: Isoform 1, Isoform 2, Isoform 3, Isoform 4, Isoform 5, Isoform 6, or Isoform 7. In some embodiments, at least one of the two or more peptide fragments comprises a sequence with at least 90% identity to a sequence selected from the group:

MGAPTLPPAWQPFLKDHRISTFKNWPFLEG,
DHRISTFKNWPFLEGCACTPERMAEAGFIH,
ACTPERMAEAGFIHCPTENEPDLAQCFF,
PTENEPDLAQCFFCFKELEGWEPDDDPIE,
FKELEGWEPDDDPIEEHKKHSSGCAFLSVK,
EHKKHSSGCAFLSVKKQFEELTLGEFLK,
QFEELTLGEFLKLDRERAKNKIAKETNNK,
RERAKNKIAKETNNKKKEFEETAEKVRRAI,
and/or
KEFEETAEKVRRAIEQLAAMD

and the polypeptide elicits an immune response or is immunostimulatory.

In some embodiments, the two or more peptide fragments comprise a sequence with at least 90% identity to

PTENEPDLAQCFFCFKELEGWEPDDDPIE
and/or
FKELEGWEPDDDPIEEHKKHSSGCAFLSVK,

and the polypeptide elicits an immune response, optionally a T-cell response.

In some embodiments, the native protein sequence is an E6 or E7 protein of a Human papillomavirus (HPV).

In some embodiments, the native protein sequence is:

MHQKRTAMFQ DPQERPRKLP QLCTELQTTI HDIILECVYC
KQQLLRREVY DFAFRDLCIV YRDGNPYAVC DKCLKFYSKI
SEYRHYCYSL YGTTLEQQYN KPLCDLLIRC INCQKPLCPE
EKQRHLDKKQ RFHNIRGRWT GRCMSCCRSS RTRRETQL;
or
MHGDTPTLHE YMLDLQPETT DLYCYEQLND SSEEEDEIDG
PAGQAEPDRA HYNIVTFCCK CDSTLRLCVQ STHVDIRTLE
DLLMGTLGIV CPICSQKP

In some embodiments, at least one of the two or more peptide fragments comprises a sequence with at least 90% identity to a sequence selected from the group:

MHGDTPTLHEYMLDLQPETTDLYCYEQLNDSSEEE,
EQLNDSSEEEDEIDGPAGQAEPDRAHYNIVTFCCK,
HYNIVTFCCKCDSTLRLCVQSTHVDIRTLEDLLMG,
and/or
IRTLEDLLMGTLGIVCPICSQKP.

In a further aspect, the invention provides a formulation comprising one or more polynucleotides encoding a native protein sequence or portion thereof and/or one or more polynucleotides encoding a polypeptide comprising two or more peptide fragments, wherein a first peptide fragment comprises a first sequence derived from a native protein sequence and wherein a second peptide fragment comprises a second sequence derived from the native protein sequence, further comprising one or more protease cleavage site sequences located between each of the two or more peptide fragments.

In some embodiments, the two or more peptide fragments comprise one or more overlapping sequences. In some embodiments, the one or more overlapping sequences are between 2 and 31 amino acids in length, optionally wherein the one or more overlapping sequences are at least 8 amino acids in length. In some embodiments, the one or more protease cleavage site sequences is an exogenous protease cleavage site, optionally a cathepsin cleavage sequence, preferably cathepsin S, more preferably an LRMK cleavage sequence. In some embodiments, the polypeptide comprises three or more peptide fragments, preferably five or more peptide fragments, more preferably ten or more peptide fragments.

In some embodiments, the formulation further comprises a pharmaceutically acceptable carrier.

In some embodiments, the formulation further comprises an adjuvant, preferably Monophosphate Lipid A (MPL), montanide, alum-based adjuvants, oil-in-water, or water-in-oil, more preferably Monophosphate Lipid A, montanide, alum-based adjuvants.

In some embodiments, the native protein sequence is the S protein of a coronavirus. In some embodiments, the coronavirus is a betacoronavirus, optionally a severe acute respiratory syndrome-related coronavirus, optionally SARS-CoV-2. In some embodiments, the coronavirus is a human coronavirus. In some embodiments, at least two of the two or more peptide fragments of the polypeptide comprise sequences derived from the S1 and/or S2 subunit of the S protein and/or wherein the portion of the native protein sequence comprises sequences derived from the S1 and/or S2 subunit of the S protein.

In some embodiments at least one of the two or more peptide fragments comprises a sequence derived from the receptor binding domain (RBD), optionally the receptor binding motif (RBM) of the S1 subunit and/or wherein the portion of the native protein sequence comprises the receptor binding domain (RBD), optionally the receptor binding motif (RBM) of the S1 subunit.

In some embodiments, at least one of the two or more peptide fragments comprises a sequence derived from the HR2 and/or HR1 domain of the S2 subunit and/or wherein the portion of the native protein sequence comprises the HR2 and/or HR1 domain of the S2 subunit.

In some embodiments the native protein sequence is survivin, chosen from any one of the following survivin isoforms: Isoform 1, Isoform 2, Isoform 3, Isoform 4, Isoform 5, Isoform 6, or Isoform 7. In some embodiments, at least one of the two or more peptide fragments comprises a sequence with at least 90% identity to a sequence selected from the group:

MGAPTLPPAWQPFLKDHRISTFKNWPFLEG,
DHRISTFKNWPFLEGCACTPERMAEAGFIH,
ACTPERMAEAGFIHCPTENEPDLAQCFF
PTENEPDLAQCFFCFKELEGWEPDDDPIE,
FKELEGWEPDDDPIEEHKKHSSGCAFLSVK,
EHKKHSSGCAFLSVKKQFEELTLGEFLK,
QFEELTLGEFLKLDRERAKNKIAKETNNK,
RERAKNKIAKETNNKKKEFEETAEKVRRAI,
and/or
KEFEETAEKVRRAIEQLAAMD

and the polypeptide elicits an immune response or is immunostimulatory.

In some embodiments, the two or more peptide fragments comprise a sequence with at least 90% identity to

PTENEPDLAQCFFCFKELEGWEPDDDPIE
and/or
FKELEGWEPDDDPIEEHKKHSSGCAFLSVK,

and the polypeptide elicits an immune response, optionally a T-cell response.

In some embodiments, the native protein sequence is an E6 or E7 protein of a Human papillomavirus (HPV).

In some embodiments, the native protein sequence is:

MHQKRTAMFQ DPQERPRKLP QLCTELQTTI HDIILECVYC
KQQLLRREVY DFAFRDLCIV YRDGNPYAVC DKCLKFYSKI
SEYRHYCYSL YGTTLEQQYN KPLCDLLIRC INCQKPLCPE
EKQRHLDKKQ RFHNIRGRWT GRCMSCCRSS RTRRETQL;
or
MHGDTPTLHE YMLDLQPETT DLYCYEQLND SSEEEDEIDG
PAGQAEPDRA HYNIVTFCCK CDSTLRLCVQ STHVDIRTLE
DLLMGTLGIV CPICSQKP

In some embodiments, at least one of the two or more peptide fragments comprises a sequence with at least 90% identity to a sequence selected from the group:

MHGDTPTLHEYMLDLQPETTDLYCYEQLNDSSEEE,
EQLNDSSEEEDEIDGPAGQAEPDRAHYNIVTFCCK,
HYNIVTFCCKCDSTLRLCVQSTHVDIRTLEDLLMG,
and/or
IRTLEDLLMGTLGIVCPICSQKP.

In a further aspect of the invention is provided a method for the immunization and/or treatment of a subject comprising administering, to the subject, the formulation of any one of the previous aspects.

A further aspect of the invention provides a composition for use in the immunization and/or treatment of a subject, wherein the composition comprises the formulation of the previous aspects, and wherein the polypeptide with the native protein sequence or portion thereof, or the one or more polynucleotides encoding the native protein sequence or portion thereof and/or the polypeptide are co-administered.

A further aspect of the invention provides a method of manufacturing a vaccine comprising expressing one or more polynucleotides encoding the native protein sequence or portion thereof and the polypeptide as described in any previous aspect, in one or more cells in vitro, and purifying the native protein sequence or portion thereof and the polypeptide. In some embodiments, the purified native protein sequence or portion thereof and the polypeptide are combined into a single formulation.

A kit for the immunization and/or treatment of a subject, comprising the native protein sequence or portion thereof of any one of the aforementioned aspects, or one or more polynucleotides encoding the native protein sequence or portion thereof, and the polypeptide of the aforementioned aspects, or one or more polynucleotides encoding the polypeptide.

A further aspect of the invention provides a method for the immunization and/or treatment of a subject comprising: administering the native protein sequence or portion thereof of the preceding aspects, or one or more polynucleotides encoding the native protein sequence or portion thereof, and administering the polypeptide of the preceding aspects, or one or more polynucleotides encoding the polypeptide.

In some embodiments, the native protein sequence or portion thereof, or one or more polynucleotides encoding the native protein sequence or portion thereof are administered simultaneously, sequentially, or separately to the polypeptide or one or more polynucleotides encoding the polypeptide.

### DESCRIPTION OF THE FIGS

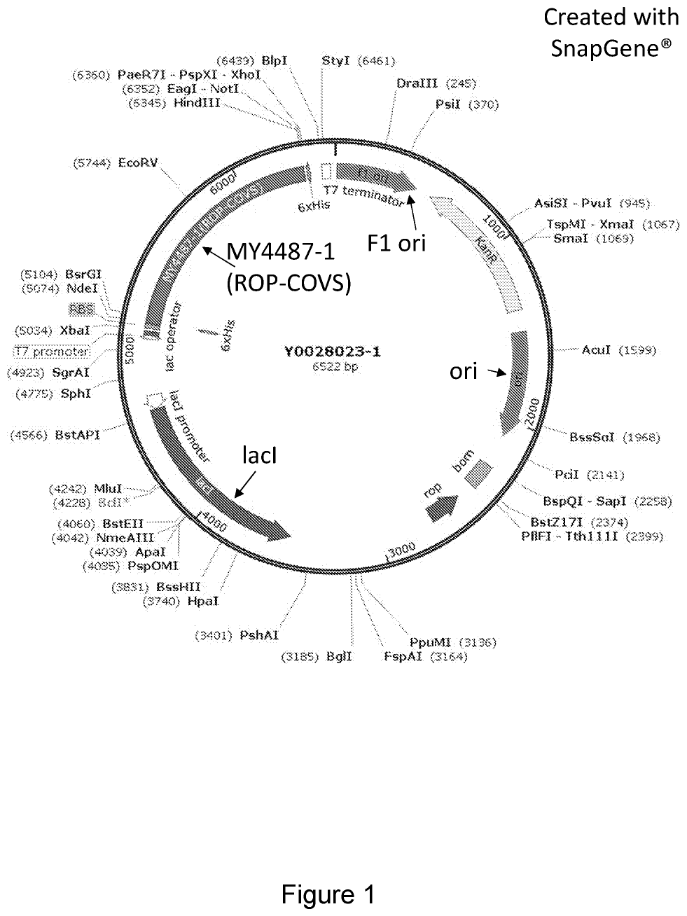

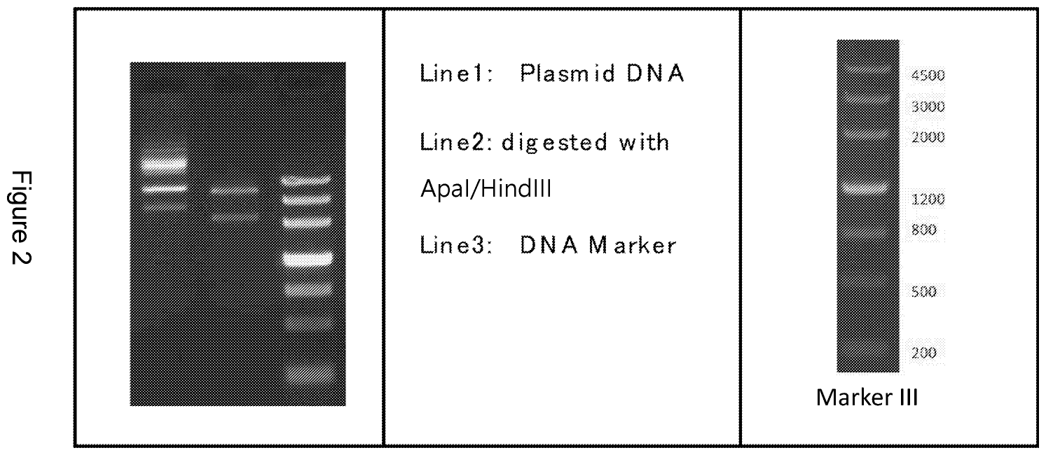

FIG. 1. Plasmid map of constructed plasmid pET30a.

FIG. 2. Electrophoretic analysis of the vector plasmid pET30a, demonstrating successful insertion of the ROP gene into E. coli.

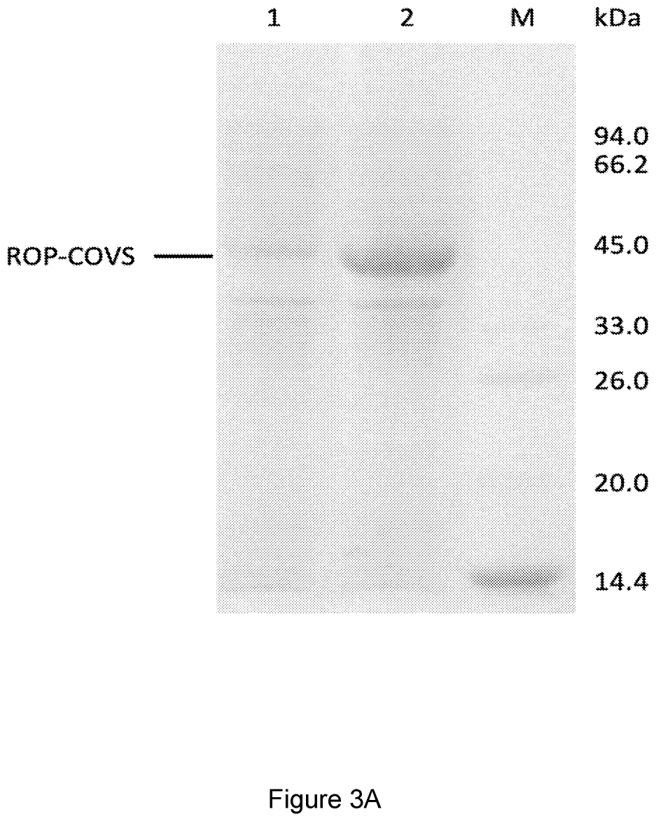

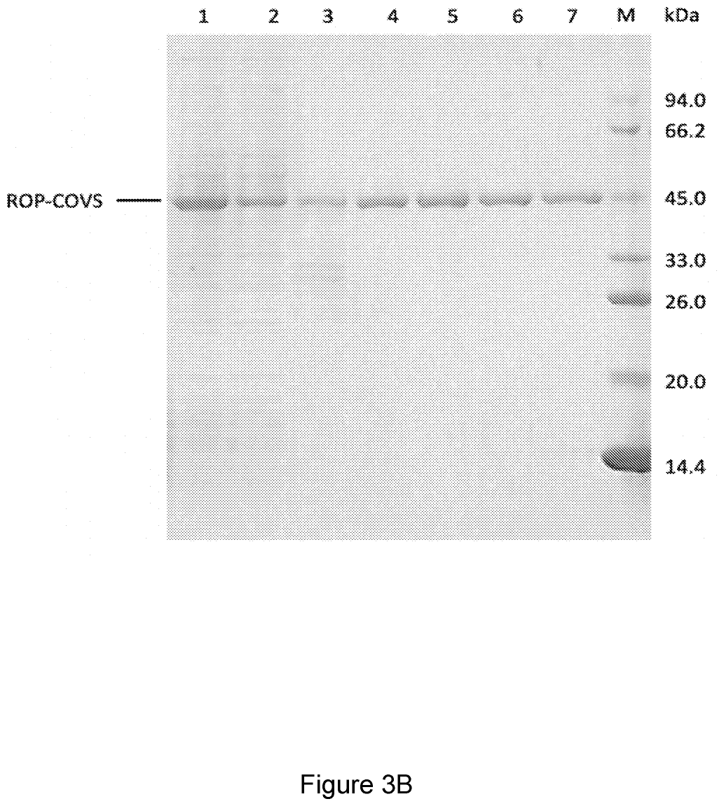

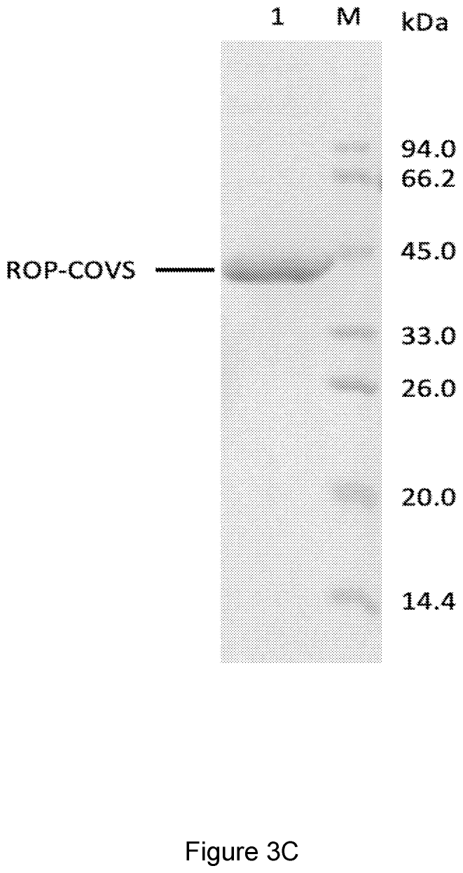

FIG. 3. SDS-PAGE analysis of:

(a) The induction rate of ROP-COVS. Lane 1 is before induction; Lane 2 is 4 hours after induction with IPTG to final concentration 0.2 mM; Lane M is the molecular weight marker (14.4-94.0 kDa). ROP-COVS is effectively induced by IPTG.

(b) Purification of ROP-COVS. Lane 1 is sample before purification; Lane 2 is the flow-through; Lane 3 is eluted with 48 mM Imidazole; Lane 4 is eluted with 78 mM Imidazole; Lane 5 & 6 is eluted with 105 mM Imidazole; Lane 7 is eluted with 138 mM Imidazole; Lane M is the molecular weight marker (14.4-94.0 kDa). Lanes 6 to 8 have over 95% purity.

(c) Refolding of ROP-COVS.

FIG. 4. An illustrative schematic of one embodiment of a polypeptide of the invention.

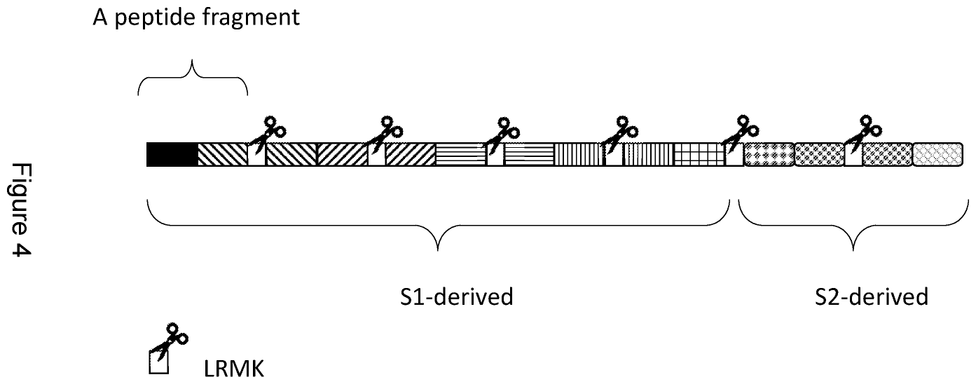

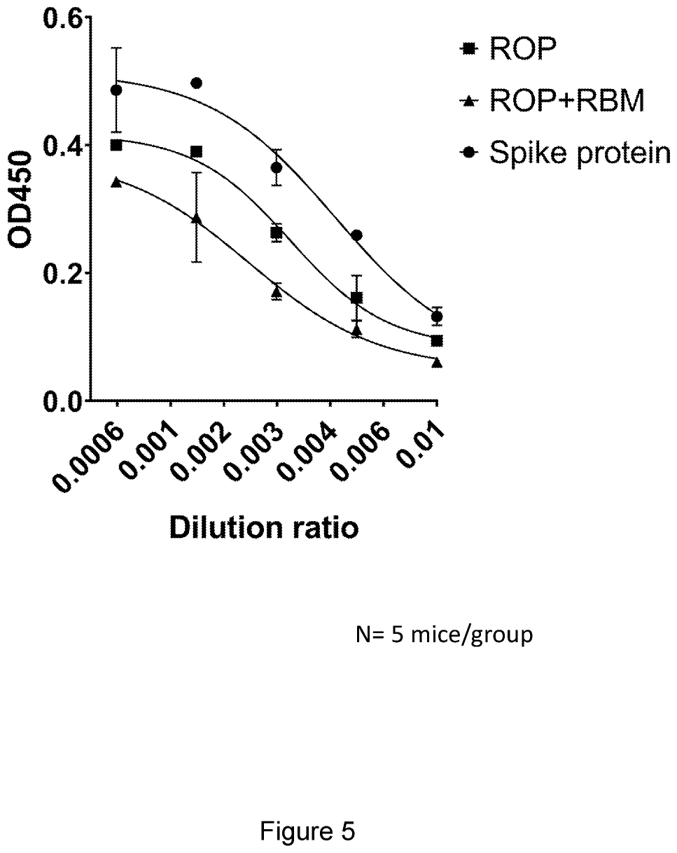

FIG. 5. Serum neutralisation data derived via surrogate RBD-ACE2 ELISA neutralisation assay.

FIG. 6. Purified IgG neutralisation data derived via surrogate RBD-ACE2 ELISA neutralisation assay.

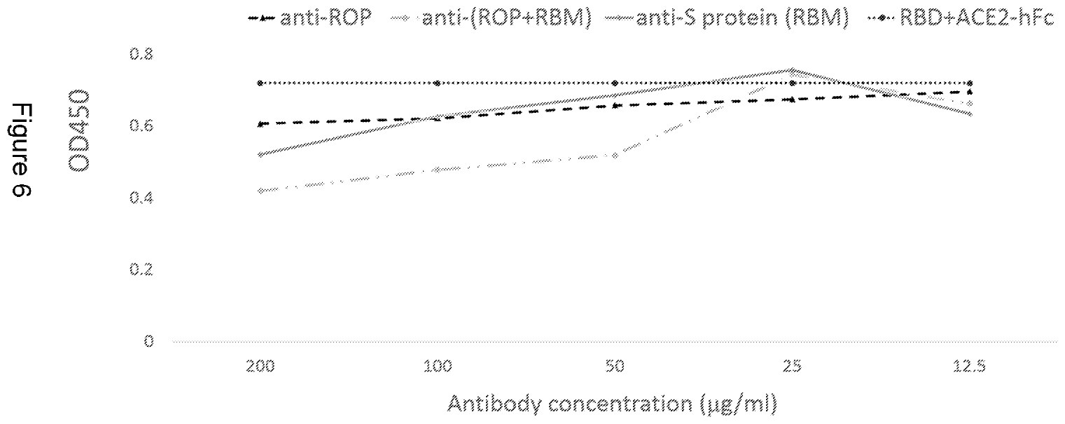

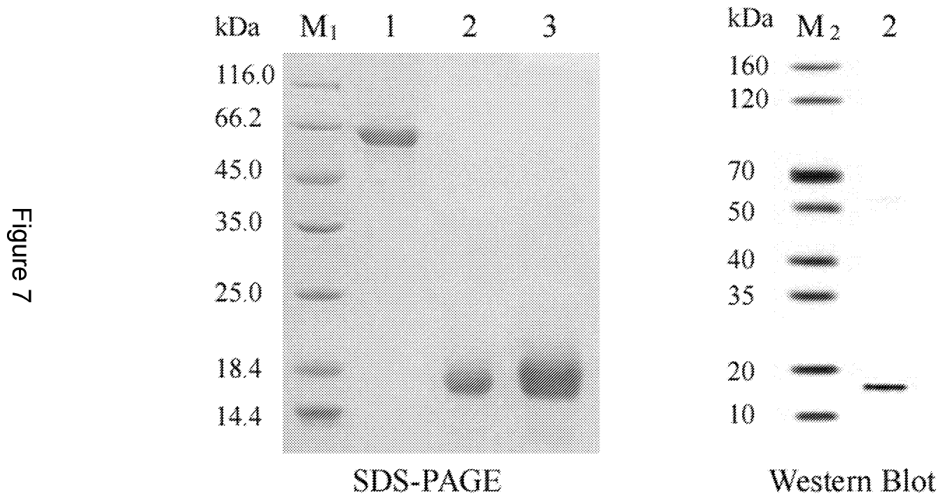

FIG. 7. An SDS-Page and Western blot showing detection of purified mouse survivin.

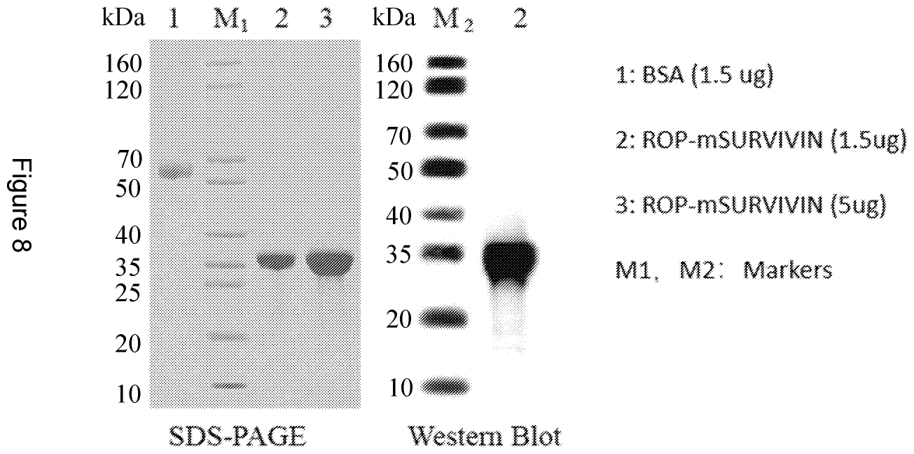

FIG. 8. An SDS-Page and Western blot showing detection of purified mouse ROP-survivin.

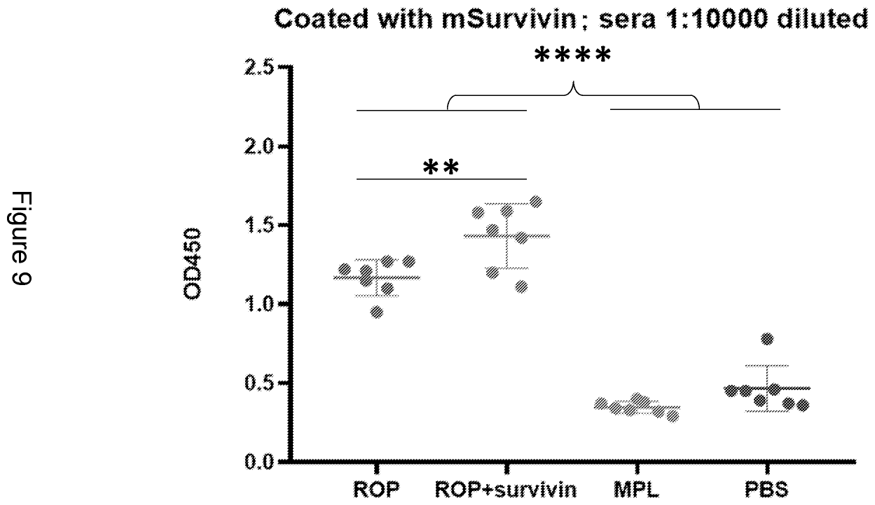

FIG. 9. A graph showing the results of an ELISA of mouse blood sera to detect antibodies binding to mouse survivin.

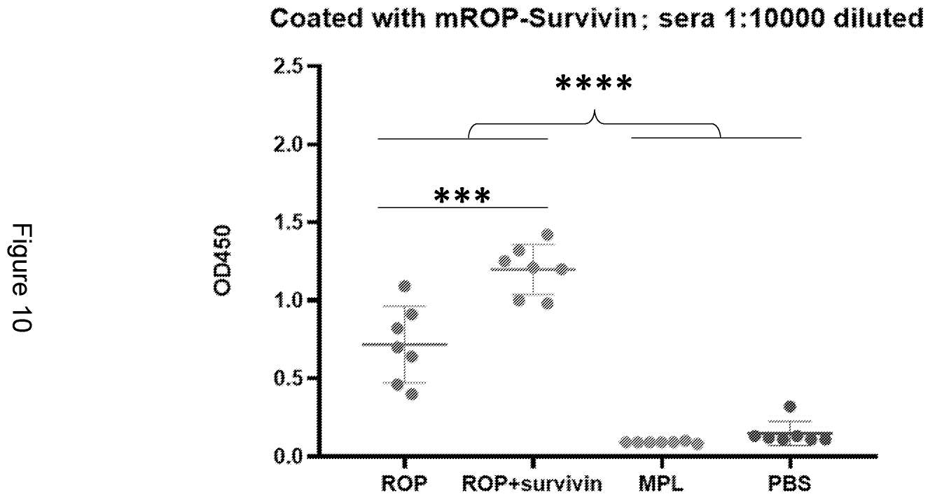

FIG. 10. A graph showing the results of an ELISA of mouse blood sera to detect antibodies binding to mouse ROP-survivin.

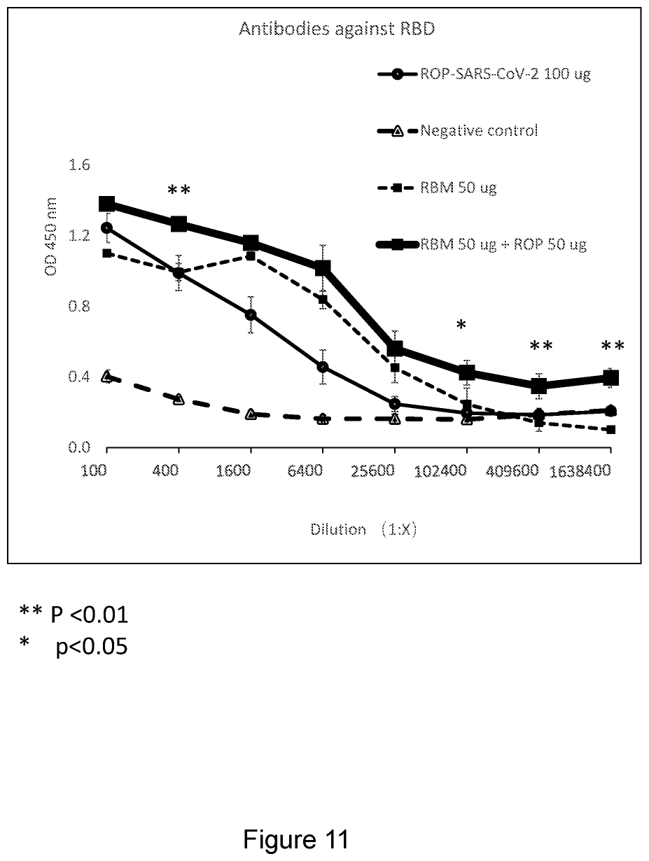

FIG. 11. A graph showing the results of an antibody titration against plate-bound RBD in an ELISA format.

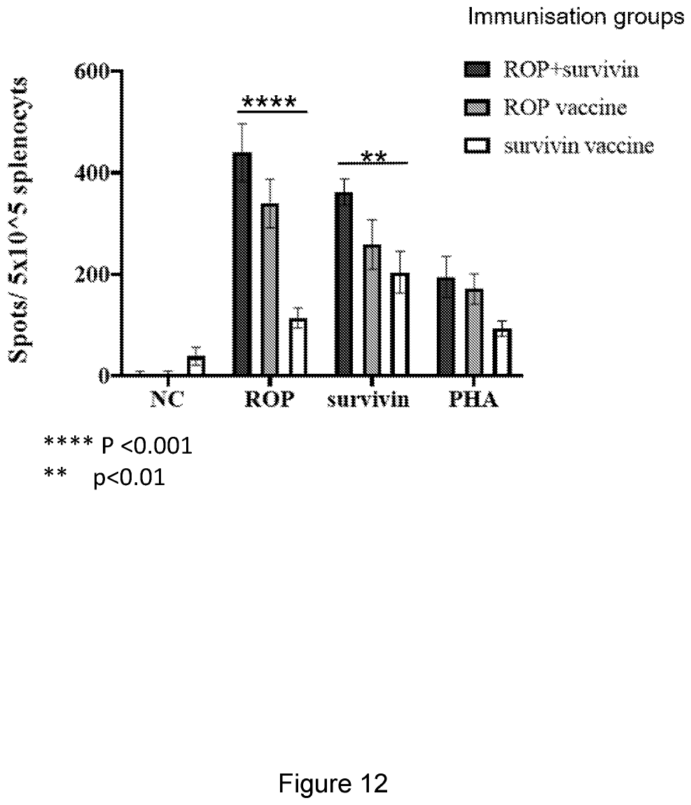

FIG. 12. A graph showing the results of an ELISPOT using splenocytes from three groups of immunised mice which have been restimulated.

### DETAILED DESCRIPTION OF THE INVENTION

Provided is a formulation for immunization and/or treatment of a subject comprising a polypeptide and a native protein sequence or a portion thereof which provides an improved efficacy of the vaccine formulation over either the polypeptide or the native protein or portion thereof alone. ‘Improved efficacy’ means that the formulation is better able to produce an antibody and/or T-cell response in a subject or produces a more pronounced antibody and/or T-cell response when administered to said subject, which can be measured by, for example, measuring the specific antibody titre and/or performing an ELISpot assay to measure T-cell response. The polypeptide comprises peptide fragments derived from the native protein, linked using protease cleavage sites to form a recombinant overlapping polypeptide which is capable of generating antibodies against the native protein sequence, and in some cases additionally stimulates CD4⁺ and CD8⁺ T-cell responses.

Oxford University Innovation, News & Publications, 28 May 2020, Oxford Vacmedix, “Oxford Vacmedix announces collaboration to develop vaccine and diagnostic tests for Covid-19” relates to a Covid vaccine project undertaken by Oxford Vacmedix UK Ltd.

CN112618707, CN112480217, CN112220920, CN112226445, and CN111671890 all relate to standard vaccine formulations relating to native proteins in the art.

Oncotarget, Vol 8, 2017, Cai et al, “Protective cellular immunity generated by cross-presenting recombinant overlapping peptide proteins” pp 76516-76524 relates to background technological information about recombinant overlapping peptide proteins.

The invention and terms used herein may be better understood with use of the following definitions.

“Recombinant” as used herein refers to any polymer, optionally a polypeptide, which is non-naturally occurring or artificially constructed, having been manufactured by gene recombination techniques in a bacterium (for example, but not limited to, an E. coli bacterium).

“Polypeptide” as used herein refers to a linear chain of amino acids linked by means of peptide bonds which is longer than a ‘peptide’ or ‘peptide fragment’, as used herein.

“Peptide” as used herein refers to a linear chain of more than one amino acid linked by means of peptide bonds which is shorter than a ‘polypeptide’ as used herein.

“Peptide fragment” as used herein refers to an amino acid chain (a “peptide”) which is a piece of a larger polypeptide. In other words, two or more peptide fragments, if fragments of the same larger polypeptide, can together form all or part of the primary sequence of the larger polypeptide. In this case, the larger polypeptide may be the recombinant polypeptide of the present invention.

“Protein” as used herein refers to a molecular entity composed primarily of one or more peptides and/or polypeptides (usually, but not essentially, having more 100 amino acids) and which has folded into, or presents as, a 3-dimensional conformation.

“Vaccine” as used herein refers to a substance capable of generating a protective immune memory against a target in a subject, wherein said subject is an animal and optionally a human. Said protective immune memory may amount to full immunity and/or a reduction in severity or symptoms of the disease associated with said target.

“Coronavirus” as used herein refers to a member of the Coronaviridae family as defined by the Coronavirus Study Group, a working group of the International Committee on Taxonomy of Viruses and as used in the Coronaviridae Study Group of the International Committee on Taxonomy of Viruses (2020) https://dx.doi.org/10.1038%2Fs41564-020-0695-z.

“Betacoronavirus” as used herein refers to a member of the Betacoronavirus genus as defined by the Coronavirus Study Group, a working group of the International Committee on Taxonomy of Viruses and as used in Coronaviridae Study Group of the International Committee on Taxonomy of Viruses (2020) https://dx.doi.org/10.1038%2Fs41564-020-0695-z. Amongst subspecies grouped within the Betacoronavirus genus are SARS-CoV, SARS-CoV-2, and MERS-CoV.

“Severe acute respiratory syndrome-related coronavirus” as used herein refers to a member of the Severe acute respiratory syndrome-related coronavirus species as defined by the Coronavirus Study Group, a working group of the International Committee on Taxonomy of Viruses and as used in Coronaviridae Study Group of the International Committee on Taxonomy of Viruses (2020) https://dx.doi.org/10.1038%2Fs41564-020-0695-z. Amongst other subspecies grouped within the Severe acute respiratory syndrome-related coronavirus species are SARS-CoV, SARS-CoV-2, SARSr-CoV BtKY72, SARSr-CoV RaTG13, SARS-CoV PC4-227, SARS-CoVGZ-02, Bat SARS CoVRf1/2004, Civet SARS CoVSz3/2003.

“Epitope” as used herein refers to a portion of a peptide fragment, peptide, polypeptide, protein, glycoprotein, lipoprotein, carbohydrate, lipid, or otherwise which is recognised by the adaptive immune system, and particularly by antibodies, B cells, and/or T cells, via receptor binding interactions.

“LRMK” as used herein refers to the Leu-Arg-Met-Lys amino acid sequence, being a cleavage site recognised by inter alia Cathepsin S. In some embodiments, a cleavable linker is provided and in some further embodiments, that linker is LRMK.

“Exogenous” as used herein means artificially introduced. It may also mean not present in the native sequence, for example the wild type (including any variants), at least in the location at which it is now artificially introduced. For example, a polypeptide may comprise two sequences which are contiguous in a native protein, and which are separated by an exogenous protease cleavage site i.e. a cleavage site which is not present in the contiguous native sequence. As another example, in the context of a polypeptide comprising peptide fragments comprising sequences derived from a SARS-CoV-2 S protein and comprising an exogenous protease cleavage site between each peptide fragment, the exogenous protease cleavage site is a cleavage site that has been artificially introduced or which is not natively found in the SARS-CoV-2 S protein at the location within the S protein amino acid sequence at which it is now located.

“Overlap” as used herein refers to a portion or ‘sub-sequence’ of an amino acid sequence which is the same, or substantially the same, in two different amino acid sequences or peptides or peptide fragments, preferably in such a way that the sub-sequence at the C-terminal end of one amino acid sequence or peptide or peptide fragment is the same as or substantially similar to the sub-sequence at the N-terminal end of another amino acid sequence or peptide or peptide fragment, and/or vice versa. Overlap may or may not be reflected in the polynucleotide sequences which encode said amino acid sequences. It will be clear to the skilled reader that ‘peptide fragments which overlap’ therefore means ‘peptide fragments having at least one overlap’.

“Identity” as used herein is the degree of similarity between two sequences, in other words the degree to which two sequences match one another in terms of residues, as determined by comparing two or more polypeptide or polynucleotide sequences. Identity can be determined using the degree of similarity of two sequences to provide a measurement of the extent to which the two sequences match. Numerous programs are well known by the skilled person for comparing polypeptide or polynucleotide sequences, for example (but not limited to) the various BLAST and CLUSTAL programs. Percentage identity can be used to quantify sequence identity. To calculate percentage identity, two sequences (polypeptide or nucleotide) are optimally aligned (i.e. positioned such that the two sequences have the highest number of identical residues at each corresponding position and therefore have the highest percentage identity) and the amino acid or nucleic acid residue at each position is compared with the corresponding amino acid or nucleic acid at that position. In some instances, optimal sequence alignment can be achieved by inserting space(s) in a sequence to best fit it to a second sequence. The number of identical amino acid residues or nucleotides provides the percentage identity, e.g. if 9 residues of a 10 residue long sequence are identical between the two sequences being compared then the percentage identity is 90%. Percentage identity is generally calculated along the full length of the two sequences being compared.

“Variant” as used in the context of a peptide, polypeptide, and/or protein herein refers to a peptide, polypeptide, and/or protein which has an amino acid sequence with at least 60%, 65%, 70%, 75%, 80%, 85%, 90%, 91%, 92%, 93%, 94%, 95%, 96%, 97%, 98% or at least 99% identity, optionally 60-100%, 65-100%, 70-100%, 75-100%, 80-100%, 85-100%, 90-100%, 91-100%, 92-100%, 93-100%, 94-100%, 95-100%, 96-100%, 97-100%, 98-100% identity, to the wild-type peptide, polypeptide, and/or protein. Where the variant differs from the wildtype, this may be due to substitution of amino acids within the sequence, and/or due to the addition or loss of amino acids from either or both ends of, or even internally within, a sequence. “Variant” may also be used in the context of a virus, (herein “a viral variant”) to refer to a virus possessing one or more mutations in its genome sequence

“Broad-acting” as used herein refers to a vaccine, therapeutic or antibody which is effective against multiple different viral species, sub-species, and/or viral variants. As an illustrative example, a broad-acting coronavirus vaccine may be effective at preventing infection across sub-species e.g. may prevent infection with SARS-CoV-2 and with SARS-CoV; in another illustrative example, a broad-acting coronavirus vaccine may be effective at preventing infection across species e.g. may prevent infection with SARS-CoV-2, SARS-CoV, MERS, HKU1, and OC43, amongst others.

“Derived from” herein and throughout means ‘identical to or substantially similar to a portion of’. A peptide fragment having a sequence derived from a protein is a peptide fragment containing an amino acid sequence which is identical to, or substantially similar to, a contiguous portion of the amino acid sequence of said protein. ‘Substantially similar’ herein and throughout means that the amino acid sequence has at least 70%, 75%, 80%, 85%, 90%, 91%, 92%, 93%, 94%, 95%, 96%, 97%, 98% or at least 99% identity, optionally 70-100%, 75-100%, 80-100%, 85-100%, 90-100%, 91-100%, 92-100%, 93-100%, 94-100%, 95-100%, 96-100%, 97-100%, 98-100% identity to the reference protein sequence, the reference SEQ ID NO, or a contiguous portion or sub-sequence thereof as will be apparent from the context. ‘At least’ herein and throughout means, in some embodiments, the recited percentage up to and including 100%. For example, ‘at least 75%’ can mean, in some embodiments, ‘75% to 100%’. Frequently, the nucleic acid sequence of a peptide fragment having a sequence derived from a protein will differ from the coronavirus protein nucleic acid sequence to a greater degree than will the amino acid sequence of the peptide fragment from the protein amino acid sequence. This is due to reasons of preparation and optimisation of expression of the polypeptide, for example codon optimisation. For the avoidance of doubt, it is the amino acid sequence of a peptide fragment which is derived from—in that it has at least 75%, 80%, 85%, 90%, 91%, 92%, 93%, 94%, 95%, 96%, 97%, 98% or at least 99% identity to a contiguous portion of—the amino acid sequence of a protein. The nucleic acid sequences may differ to a greater extent and may have a lower sequence identity due to the inherent redundancy of the genetic code for amino acids. In the definition of ‘derived from’ above, the protein referred to may be the native protein sequence, optionally the wild-type native protein sequence.

‘At least’ herein and throughout means, in some embodiments, the recited number of peptide fragments up to and including the total number of peptide fragments present in the polypeptide. For example, in a polypeptide with 14 peptide fragments ‘at least two peptide fragments’ would mean, in some embodiments, ‘two to 14 peptide fragments’, or any number in between.

An “overlapping sequence” is a portion or sub-sequence of an amino acid sequence which is present in two or more peptide fragments of the polypeptide of the present invention. In some embodiments, the C-terminal end of one peptide fragment comprises an amino acid sequence which is the same as or substantially similar to the amino acid sequence at the N-terminal end of another peptide fragment. That means that where there is an overlapping sequence, there must be at least one portion of a peptide fragment which is the same on at least two peptide fragments. In some embodiments, the overlapping sequence is 2 to 40 amino acids in length, so each overlapping portion of a peptide fragment is 2 to 40 amino acids. In some embodiments, the overlapping sequence is 2 to 31 amino acids in length. In other embodiments, the overlapping sequence is 4 to 30 amino acids in length. In other embodiments, the overlapping sequence is 6 to 20 amino acids in length. In preferred embodiments, the overlapping sequence is 8 to 17 amino acids in length. In some embodiments, overlapping sequences are 8, 9, 10 or 11 amino acids in length. In some embodiments, overlapping sequences are 12 amino acids in length. In other embodiments, overlapping sequences are 13 amino acids, 14 amino acids, 15, 16, or 17 amino acids in length. In a most preferred embodiment, the overlapping sequence is at least 8 amino acids in length for the generation of a cytotoxic T lymphocyte (‘CTL’) (CD8+ T cell) response and/or at least 12 amino acids in length for the generation of a T helper cell (CD4+ T cell) response.

In one embodiment, the polypeptide of the invention comprises peptide fragments comprising a sequence which overlaps with that of one other peptide fragment within the polypeptide—for example, by means of its N-terminal sequence or its C-terminal sequence. In another embodiment, the polypeptide of the invention comprises peptide fragments comprising a sequence which overlaps with those of two other peptide fragments within the polypeptide—for example, by means of its N-terminal sequence and its C-terminal sequence. In some embodiments, the polypeptide of the invention additionally comprises one or more peptide fragment(s) which comprise a sequence which does not overlap with the sequence of any other peptide fragment contained within the polypeptide.

Any one peptide fragment may be 2 to 55 amino acids in length, more preferably 8 to 50 amino acids in length, more preferably 12 to 45 amino acids, more preferably 20 to 40 amino acids in length. In a preferred embodiment, each peptide fragment is 25 to 40 amino acids long, more preferably 28 to 38 amino acids long, even more preferably 29 to 37 amino acids long. In preferred embodiments, each peptide fragment is 29, 30, 31, 32, 33, 34, 35, 36, or 37 amino acids in length.

In all embodiments of the invention, peptide fragments are linked together in tandem to form the polypeptide by means of at least one protease cleavage site sequence located between each linearly adjacent peptide fragment. ‘Linearly adjacent’ is taken here to mean peptide fragments which are immediately sequential in terms of secondary structure or amino acid sequence. Accordingly, one or more protease cleavage site sequences separate each peptide fragment. Peptide fragments are connected by means of one or more protease cleavage site sequences. In one embodiment of the invention, two or more peptide fragments are linked together in tandem to form the polypeptide by means of at least one protease cleavage site sequence located between each linearly adjacent peptide fragment. In another embodiment, three or more peptide fragments are linked together in tandem to form the polypeptide by means of at least one protease cleavage site sequence located between each linearly adjacent peptide fragment. In another embodiment, 4 to 30, 5 to 20 peptide fragments, more preferably 10 to 15, 11 to 14, 12, or 13 peptide fragments are linked together in tandem to form the polypeptide by means of at least one protease cleavage site sequence located between each linearly adjacent peptide fragment.

Where a dosage is expressed in ‘μg·kg⁻¹’, this is intended to mean the mass of the agent in micrograms per mass of the subject in kilograms. It will be clear to the skilled reader, therefore, that mg·kg⁻¹ means the mass of the agent in milligrams per mass of the subject in kilograms. The agent may be any of those listed herein i.e. the polypeptide, or the native protein sequence or portion thereof. Said therapeutic and/or prophylactic polypeptide and/or native peptide sequence or fragment thereof may be provided to a mammalian subject, preferably a human. In addition, polynucleotides encoding any of the above are also envisaged for administration to a mammalian subject, preferably a human.

In a first aspect, the present invention provides a formulation for immunizing and/or treating a subject comprising a polypeptide comprising two or more peptide fragments, wherein a first peptide fragment comprises a first sequence derived from a native protein sequence and wherein a second peptide fragment comprises a second sequence derived from the native protein sequence, further comprising one or more protease cleavage site sequences located between each of the two or more peptide fragments; and the native protein sequence or a portion thereof. In some embodiments, the formulation is alternatively or additionally for vaccinating a subject, and is a vaccine formulation. The first sequence and second sequence, and any further sequences of the polypeptide may be variants of all or part of the native protein sequence as outlined above. The native protein itself may be slightly modified compared to the wild-type sequence to, for example, improve the immunogenicity thereof. Rather than providing the entire native protein sequence, the skilled person will appreciate that in some embodiments a portion thereof may be provided. Such a portion thereof may comprise a known antigenic portion, or otherwise a functionally relevant portion, meaning that the provided portion is known to play a key role in the function of the native protein sequence, or may otherwise be key for immune recognition of the native protein sequence. As such, the portion of the native protein sequence may comprise a known epitope for generating an immune response against the native protein sequence. Without wishing to be bound by theory, the ROP stimulates a strong T cell response including a CD4⁺ and CD8⁺ T cell response, and CD4⁺ helps to stimulate the development of antibodies. An interaction between T cells and B cells stimulates a strong B cell response, as the cytokines released from the T cells stimulates the B cell response in a non-linear fashion. By exposing the immune system to the two antigenic proteins, an amplification of the response occurs owing to the differing but simultaneous activation of multiple pathways of the immune system.

To illustrate the first aspect, the formulation may comprise a polypeptide comprising two or more peptide fragments each comprising a sequence derived from a native protein sequence, wherein the native protein sequence is the spike (or ‘S’) protein of the SARS-CoV-2 coronavirus. As such, the formulation comprises a polypeptide with two or more peptide fragments, wherein the first peptide fragment comprises a first sequence derived from the native protein sequence, and wherein the second peptide fragment comprises a second sequence derived from the native protein sequence, each separated by a protease cleavage site sequence. The formulation additionally comprises the native protein sequence or a portion thereof. In this illustration, that means that as well as the polypeptide outlined above, the formulation additionally comprises the spike protein or a portion thereof. As a further illustration the portion thereof might be, for example, the receptor binding motif of the spike protein, which is known to play a critical role in the entry of the coronavirus into a host cell.

The skilled person will appreciate that such a formulation can be administered in a variety of ways. The most common administration route is by injection, although oral delivery and nasal spray delivery are also envisaged. When injected, delivery may be subcutaneous, intravenous, intramuscular, intraperitoneal, or intradermal.

In some embodiments, the two or more peptide fragments comprise one or more overlapping sequences. As an illustrative example, the polypeptide may comprise two peptide fragments derive from the native protein sequence, where the first peptide fragment comprises amino acid residues 1 to 10, and the second peptide fragment comprises amino acid residues 5 to 15, thus the polypeptide has an overlapping sequence comprising sequences 5 to 10, which are present in both fragments. Polypeptides comprising these overlapping sequences may be referred to as recombinant overlapping polypeptides (ROPs). ROPs have been shown to provide advantages over conventional vaccines (see Cai et al., 2017, WO2007125371, and WO2016095812).

The polypeptide comprises at least two or more peptide fragments. In some embodiments it may comprise three or more peptide fragments, four or more peptide fragments, five or more peptide fragments, six or more peptide fragments, seven or more peptide fragments, eight or more peptide fragments, nine or more peptide fragments, ten or more peptide fragments, eleven or more peptide fragments, or twelve or more peptide fragments. In some embodiments it may comprise more than twelve peptide fragments. It will be understood that in cases where there are three or more peptide fragments, each of these will have an amino acid sequence which is a variant of or derived from the native protein sequence. The sequence may be identical between peptide fragments or may be different between each peptide fragment. As an illustrative example, the first peptide fragment may have a first comprising residues 1 to 10 from e.g. survivin isoform 1, the second peptide fragment may have a second sequence comprising residues 11 to 20, and the third peptide fragment may have the first sequence comprising residues 11 to 20.

In some embodiments, the polypeptide may comprise multiple overlapping sequences. As an illustrative example, the first peptide fragment may comprise residues 1 to 10, the second peptide fragment may comprise residues 5 to 15, and the third peptide fragment may comprise residues 11 to 20. Thus, in the illustrative example, there are two overlapping sequences in the polypeptide, specifically residues 5 to 10 in the first and second peptide fragments, and 11 to 15 in the second and third peptide fragments. In addition, or alternatively, there may be one or more overlapping sequences, but not all of the peptide fragments need contain an overlapping sequence. As an illustrative example, the first and second peptide fragments may contain an overlapping sequence defined by residues 5 to 10, but the third peptide fragment may comprise residues 16 to 25, and thus not overlap with either. In some embodiments the polypeptide may comprise 2, 3, 4, 5, 6, 7, 8, 9, 10, 11, 12, 13, 14, 15, 16, 17, 18, 19, 20, 21, 22, 23, 24, 25 or more overlapping sequences.

Any number of overlaps may be present and this is only limited by the number and size of the peptide fragments of the polypeptide.

In some embodiments, the polypeptide may comprise a peptide fragment with a sequence having partial sequence identity to the wild-type native protein sequence (e.g. any of the isoforms listed above, or their homologues). As an illustrative example, at least one peptide fragment may comprise a sequence with at least 99% identity to the relevant part of the native protein sequence. Alternatively, at least one peptide fragment may comprise a sequence with at least 98%, 97%, 96%, 95%, 94%, 93%, 92%, 91%, 90%, 80%, 70%, 60%, 50%, 40%, 30%, 20%, or 10% identity to the relevant part of the native protein sequence. ‘Relevant part’ means the contiguous string of residues of the native protein sequence on which the peptide fragment in question is based. As an illustrative example, if the peptide fragment comprises a sequence with at least 90% identity to residues 1 to 10 of the native protein sequence, then 9 of the 10 residues will be identical to residues 1 to 10 of the native protein sequence, and one will be different. The skilled reader will understand that any residues can be interchanged provided the percentage identity is intact. The skilled reader will further understand that a lower percentage identity is acceptable provided key residues are maintained.

Each of the two or more peptide fragments can be any length in terms of amino acids. Each of the two or more peptide fragments could be 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35 or more amino acids in length. The overlap between peptide fragments (i.e. the overlapping sequences) may be limited by the length of the peptide fragment, and these overlapping sequences may be at least 2, 3, 4, 5, 6, 7, 8, 9, 10, 11, 12, 13, 14, 15 or more amino acids in length. In some embodiments, the one or more overlapping sequences are between 2 and 31 amino acids in length, optionally wherein the one or more overlapping sequences are at least 8 amino acids in length.

The two or more peptide fragments of the polypeptide may comprise one or more sequences which cover the whole sequence of the protein. As an illustrative example, where the native protein sequence has a sequence of 142 amino acids, the polypeptide may comprise two peptide fragments, the first peptide fragment having a sequence derived from residues 1 to 71 of the native protein sequence, the second fragment having a sequence derived from residues 72 to 142 of the native protein sequence. The skilled reader will understand that any number of fragments may be used to cover the whole of the native protein sequence upon which the polypeptide is based. As a further illustrative example, the polypeptide may comprise three polypeptide fragments, the first peptide fragment having a first sequence derived from residues 1 to 71 of the native protein sequence, the second peptide fragment having a second sequence derived from residues 72 to 142 of the native protein sequence, and the third peptide fragment having a third sequence derived from residues 50 to 120 of the native protein sequence.

Such a polypeptide may comprise any number of overlapping sequences, it can comprise peptide fragments of any length, and the polypeptide sequence can be any length provided the peptide fragments are derived from the native protein sequence or variants thereof as outlined above. Each peptide fragment of the polypeptide may be a different sequence derived from the native protein sequence.

In some embodiments, the polypeptide and/or the native protein sequence or portion thereof of the invention is immunostimulatory. In some embodiments, one or more of the peptide fragments of the polypeptide of the invention are immunostimulatory. In some embodiments, one or more of the sequences comprised within the peptide fragments of the polypeptide of the invention are immunostimulatory. ‘Immunostimulatory’ as referred to herein means stimulates, motivates, causes, and/or produces an immune response when administered to a subject. In preferred embodiments, said immune response comprises an adaptive immune response. In some embodiments, said adaptive immune response comprises the generation of antibodies against the polypeptide and/or against one or more peptide fragments and/or sequences comprised therein. In other embodiments, said adaptive immune response comprises the activation and/or proliferation of CD8+ and/or CD4+ T cells. In some embodiments, said adaptive immune response comprises the generation of antibodies against the polypeptide and/or against one or more peptide fragments and/or sequences comprised therein and, further, the activation and/or proliferation of CD8+ and/or CD4+ T cells.

One or more protease cleavage site sequences are located between each of the two or more peptide fragments of the polypeptide of the present invention. In a preferred embodiment, the one or more protease cleavage site sequences are cleavage site sequences of a protease present in the target or host or subject or patient to whom the polypeptide is administered, such that the polypeptide may be cleaved within the host into its peptide fragments. In some embodiments, the one or more protease cleavage site sequences is an exogenous protease cleavage site, optionally a cathepsin cleavage sequence, preferably cathepsin S, more preferably an LRMK cleavage sequence. Said protease may act extracellularly or, more preferably, intracellularly. Said protease may be a non-host protease delivered in combination with the polypeptide or its encoding polynucleotide. More preferably, said protease is a host protease. As an illustrative example, the polypeptide may comprise six peptide fragments, each separated by one or more protease cleavage sites, wherein the one or more protease cleavage sites comprise four cathepsin S cleavage sites, preferably LRMK protease cleavage sites.

In an embodiment, the two or more peptide fragments comprise at least one peptide fragment comprising one or more linear antibody epitope(s) of the native protein sequence, and comprising a protease cleavage site sequence located between each peptide fragment. The exogenous cleavage site located between each peptide fragment is useful because it allows peptide fragments to be liberated in a desired manner. In some embodiments the exogenous protease cleavage site sequence is for an intracellular protease and thereby allows peptide fragments to be liberated from the polypeptide intracellularly. In some embodiments at least one linear antibody epitope is a neutralising epitope. In some embodiments, the two or more peptide fragments comprise amino acid sequences which overlap. This may allow that, for example, while one or more linear antibody epitope sequence is comprised wholly within one peptide fragment, other peptide fragment(s) comprise partial sequences of said linear antibody epitope. In some embodiments, at least one peptide fragment comprises one or more CD4+ and/or CD8+ T cell epitope(s) of the native protein sequence. A peptide fragment may comprise one epitope only (whether a linear antibody epitope or CD4+ or CD8+ T cell epitope). Equally, a peptide fragment may comprise some or all of two epitopes, for example some or all of a linear antibody epitope and some or all of a T cell epitope. A peptide fragment may comprise no epitope.

CD8⁺ T cells (also ‘Cytotoxic T Lymphocytes’, ‘CTLs’) target and lyse diseased and/or infected cells. Traditionally, MHC class I molecules are understood to present fragments of intracellular origin for CD8+ T cell recognition and activation; for example, a cancerous cell may present fragmented products of proteasomal digestion of aberrantly expressed, intracellular proteins on MHC class I cells. CD4+ T cells assist in the activation and expansion of other immune cells, including T cells and B cells. Traditionally, MHC class II molecules are understood to present, to CD4+ T cells, fragments of extracellular origin which have been internalised by antigen-presenting cells for presentation. More recently, cross-presentation has been shown known to occur in addition to these traditional pathways, whereby internalised extracellular fragments may be presented on MHC class I molecules. In some embodiments at least one peptide fragment of the polypeptide comprises one or more CD4+ T cell epitope(s) and/or one or more CD8+ T cell epitope(s) of the native protein sequence.

The peptide fragments of the present invention, having been cleaved by a protease, may be processed and presented, for example via MHC class I and class II molecules, to cells of the immune system. Amino acid sequences derived from the peptide fragments of the present invention stimulate CD8⁺ and CD4+ T cells via their presentation via MHC class I and class II molecules, respectively.

In some embodiments, the polypeptide of the invention is very effective at simulating the T cell response. In some embodiments, the polypeptide stimulates the CD8+ T cell response. In some embodiments, the polypeptide stimulates the CD4+ T cell response. In some embodiments, the polypeptide of the invention stimulates both the CD8+ and CD4+ T cell response. In some embodiments, the two or more fragments of the polypeptide comprises at least one fragment comprising a T-cell epitope.

In some embodiments, both the polypeptide and the native protein sequence stimulate the CD4+ and CD8+ T cell response. The polypeptide of the invention comprises overlapping peptide fragments, which further strengthens the T cell response (Zhang et al., 2009). Further, the use of overlapping peptides more comprehensively represents the range of potential T cell epitopes.

Genetic variation in T cell receptor and MHC repertoires within a population mean there may exist population-wide variation in the sequences presented to and/or recognised by CD4+ and/or CD8⁺ T cells. The multiple and overlapping peptide fragments of the present invention compensate this variation via the ability to tile, or provide greater coverage of, one or more epitopes and by providing alternative options for immune recognition, reducing any need for HLA typing.

In some embodiments, the polypeptide and/or the native protein sequence or portion thereof is provided as a polynucleotide (either DNA, RNA, or a mixture of both) encoding said polypeptide. For the avoidance of doubt, the polypeptide and native protein sequence or portion thereof may be provided on a single polynucleotide, or different polynucleotides. Such a polynucleotide can be used in place of the polypeptide and/or native protein sequence or portion thereof in any of the methods of the invention. For example, a polynucleotide encoding the polypeptide can be co-administered with a polynucleotide encoding the native protein sequence to a subject, and once administered will cause expression of the polypeptide and native protein sequence of the invention such that effectively both the polypeptide and native protein sequence have been administered to the subject.

In some embodiments, the formulation further comprises a pharmaceutically acceptable carrier. In the context of a formulation, the polypeptide and the native protein sequence or portion thereof are mixed within the same volume of the pharmaceutically acceptable carrier. However, in some aspects of the invention, the polypeptide and the native protein sequence or portion thereof are provided in separate volumes of the pharmaceutically acceptable carrier, and are intended to be administered simultaneously, separately or sequentially. Where the polypeptide and native protein sequence or portion thereof are provided in separate volumes, it will be understood that any of the embodiments described in the formulation above are equally applicable to each constituent in its separate volume. In addition, and for the avoidance of doubt, the separate volumes may alternatively or additionally include one or more polynucleotides encoding the polypeptide and/or native protein sequence or portion thereof.

The polypeptide and native protein of the invention and/or the polynucleotide of the invention may be administered to a subject by means of a delivery vehicle. In one embodiment, the pharmaceutically acceptable delivery vehicle is a viral vector, for example—but not limited to—an adenovirus, an adeno-associated virus, MVA, HSV. In another embodiment, the pharmaceutically acceptable delivery vehicle is a bacterial vector, for example—but not limited to—Listeria spp., Salmonella spp. In another embodiment, the pharmaceutically acceptable delivery vehicle is a plasmid, a nanoparticle, a lipoparticle, a polymeric particle, or a virus-like particle.

In one embodiment, the composition or pharmaceutical composition optionally comprises one or more pharmaceutically acceptable carriers (or excipients). Examples of such suitable excipients for the different forms of pharmaceutical compositions described herein may be found in the “Handbook of Pharmaceutical Excipients”, 2nd Edition, (1994), Edited by A Wade and PJ Weller. The composition or pharmaceutical composition may comprise one or more additional components. In one embodiment, the carrier is suitable for injectable delivery. In another embodiment, the carrier is suitable for pulmonary delivery. In another embodiment, the carrier is suitable for oral delivery.

In some embodiments, the formulation further comprises an adjuvant, preferably Monophosphate Lipid A (MPL), montanide, alum-based adjuvants, oil-in-water, or water-in-oil, more preferably Monophosphate Lipid A, montanide, alum-based adjuvants.

In some embodiments, the concentration of the polypeptide is between 10 to 10000 μg·kg⁻¹ and the concentration of the native protein sequence or portion thereof is between 10 to 10000 μg·kg⁻¹. Concentration in this context is sometimes referred to as the dose concentration or simply the ‘dose’ and each term can be used interchangeably. In practical terms, this unit means that the amount of polypeptide or native protein sequence or portion thereof (in μg) administered to a subject is adjusted based upon that subject's weight (in kg). For example, if a subject weights 100 kg, then the amount of polypeptide and/or native protein sequence or portion thereof provided to that subject will be between 1000 μg and 1000000 μg.

In some embodiments, the native protein sequence is the S protein of a coronavirus. In some embodiments, the coronavirus is a betacoronavirus, optionally a severe acute respiratory syndrome-related coronavirus, optionally SARS-CoV-2. In some embodiments, the coronavirus is a human coronavirus. In some embodiments, at least two of the two or more peptide fragments of the polypeptide comprise sequences derived from the S1 and/or S2 subunit of the S protein and/or wherein the portion of the native protein sequence comprises sequences derived from the S1 and/or S2 subunit of the S protein.

In some embodiments at least one of the two or more peptide fragments comprises a sequence derived from the receptor binding domain (RBD), optionally the receptor binding motif (RBM) of the S1 subunit and/or wherein the portion of the native protein sequence comprises the receptor binding domain (RBD), optionally the receptor binding motif (RBM) of the S1 subunit.

In some embodiments, at least one of the two or more peptide fragments comprises a sequence derived from the HR2 and/or HR1 domain of the S2 subunit and/or wherein the portion of the native protein sequence comprises the HR2 and/or HR1 domain of the S2 subunit.

In one embodiment of the invention, the polypeptide comprises two or more peptide fragments, at least one—optionally more than one—of which comprises a sequence derived from the S protein of a severe acute respiratory syndrome-related coronavirus, optionally SARS-CoV-2. In an embodiment of the invention, at least one—optionally more than one—of said two or more peptide fragments comprises a sequence derived from the S1 subunit. In an embodiment, at least one—optionally more than one—of said two or more peptide fragments comprises a sequence derived from the RBD of the S1 subunit. In some embodiments, at least one—optionally more than one—of said two or more peptide fragments comprises a sequence derived from the receptor binding motif (‘RBM’) of the RBD. In another preferred embodiment of the invention, at least one—optionally more than one—of said two or more peptide fragments comprises a sequence derived from the S2 subunit. In an embodiment, at least one—optionally more than one—of said two or more peptide fragments comprises a sequence derived from the heptad repeat 2 (‘HR2’) domain of the S2 subunit. In another embodiment, at least one—optionally more than one—of said two or more peptide fragments comprises a sequence derived from the heptad repeat 1 (‘HR1’) domain of the S2 subunit. The skilled person will appreciate that ‘derived from’ carries the meaning outlined above.

It will be understood that there exist multiple viral variants and/or strains of Severe acute respiratory syndrome-related coronavirus subspecies such as SARS-CoV-2 (e.g. B.1.1.7, B.1.351, P.1, B.1.427, B.1.429), and that new viral variants will continue to emerge. The peptide fragments of the present invention may derive from any, or multiple, of such viral variant strains. The amino acid sequence of the peptide fragments can be readily adjusted to represent new mutations and variants in order to provide immune protection to a subject receiving the fusion protein of the present invention against emergent viral variant strains.

The RBD is a domain of the S1 subunit of S proteins which binds to a host receptor. The RBD of SARS-CoV-2 binds strongly to angiotensin-converting enzyme 2 (ACE2) of at least humans and bats (Tai, W., et al. (2020)). The RBD of SARS-CoV binds ACE2. The RBD of MERS-CoV binds dipeptidyl peptidase 4 (DPP4). The RBD of SARS-CoV-2 may be represented as SEQ ID NOs: 15 or 16 and in some embodiments, the RBD comprises residues 318 to 541 of SARS-CoV-2 S proteins (Yi, C., et al. (2020)). In other embodiments, the RBD may comprise residues 319 to 529, 331-524, or 336-516 of SARS-CoV-2 S proteins (Shang, J., et al. (2020); Tai, W., et al. (2020); Lan, J., et al. (2020)). The RBD of SARS-CoV may comprise residues 306-527 and/or 318-510 of SARS-CoV S proteins; the RBD of MER-CoV S may comprise residues 377-588 of MERS-CoV S proteins (Yi, C., et al. (2020); Tai, W., et al. (2020)). The skilled person will appreciate that the boundaries of the RBD as defined by residue numbers may vary slightly and as seen above. Thus, the RBD may comprise the amino acid sequences having residues defined above or variants thereof.

The RBM is a motif of the S1 subunit of S proteins, and within the RBD, which binds to a host receptor. The RBM of SARS-CoV-2 may be represented as SEQ ID NO: 17 and, in some embodiments, the RBM of SARS-CoV-2 comprises residues 438-506 of SARS-CoV-2 S proteins (Lan, J., (2020)). The skilled person will appreciate that the boundaries of the RBM as defined by residue numbers may vary slightly. Thus, the RBM may comprise the amino acid sequences having residues defined above or variants thereof.

HR1 is a heptad repeat which forms a 6-helical bundle (6HB) with the HR2 heptad repeat which brings the viral envelope into close proximity with host cell membranes for fusion. HR1 may be represented as SEQ ID NO: 35 and in some embodiments comprises residues 910 to 988 of SARS-CoV-2 S proteins. In other embodiments, HR1 may comprise residues 912 to 984 or 920 to 970 of SARS-CoV-2 S proteins (Xia, S., et al. (2020)). The HR1 of SARS-CoV may comprise residues 902 to 952 of SARS-CoV S proteins. The skilled person will appreciate that the boundaries of the HR1 as defined by residue numbers may vary slightly. Thus, the HR1 may comprise the amino acid sequences having residues defined above or variants thereof.

HR2 is a heptad repeat which forms a 6-helical bundle (6HB) with the HR2 heptad repeat which brings the viral envelope into close proximity with host cell membranes for fusion. HR2 may be represented as SEQ ID NO: 19 and in some embodiments comprises residues 1159-1211 of SARS-CoV-2 S proteins. In other embodiments, HR2 may comprise residues 1163-1202 of SARS-CoV-2 S proteins (Xia, S., et al. (2020)). The HR2 of SARS-CoV may comprise residues 1145-1184 of SARS-CoV S proteins. The skilled person will appreciate that the boundaries of the HR2 as defined by residue numbers may vary slightly. Thus, the HR2 may comprise the amino acid sequences having residues defined above or variants thereof.

The HR1 and HR2 regions are key functional regions of the S2 subunit of the coronavirus S protein, being essential for fusion of the viral envelope with the host cell membrane. Antibodies which bind to or close to, i.e. are directed against, key functional regions are able to block, interfere with, or prevent the viral function of said regions, sterically or otherwise. By providing one or more sequences of HR1 and/or HR2, the polypeptide of the present invention stimulates the generation of neutralising and/or broad-acting antibodies against HR1 and/or HR2 which preclude viral entry into host cells. This is distinct from any use of isolated amino acid sequences of HR1 and/or HR2 (whether in native or stapled form) to directly inhibit coronavirus entry via direct binding of said HR1 and/or HR2 sequences to the coronavirus' own HR1 and/or HR2 and subsequent preclusion of formation of the coronavirus-associated HR1-HR2 6HB, as described in e.g. CN111560054 and CN111732637.

The RBD and RBM are key functional regions of the S1 subunit of the coronavirus S protein, being essential for coronavirus-host receptor binding. Antibodies which bind to, and are directed against, key functional regions are able to block, interfere with, or prevent the viral function of said regions, sterically or otherwise. By providing one or more sequences of the RBD, optionally further the RBM, the polypeptide of the present invention stimulates the generation of neutralising and/or broad-acting antibodies against the RBD, and optionally the RBM, which preclude binding of viral S1 to host receptors.

In some embodiments where the native protein sequence is that of the spike protein or a portion thereof, the two or more peptide fragments of the present invention may comprise any one of the sequences SEQ ID NOs 1 to 12, as detailed below, or a variant thereof. In another embodiment, any one of the three or more peptide fragments of the present invention may comprise any one of the sequences SEQ ID NOs 1 to 12, as detailed below, or a variant thereof. In another embodiment, the polypeptide comprises one or more, two or more, three or more, four or more, five or more, six or more, seven or more, eight or more, nine or more, ten or more, eleven or more, or twelve of the sequences SEQ ID NOs 1 to 12, as detailed below, or a variant thereof:

PF1 (30 aa):
(SEQ ID NO: 1)
SVLYNSASFSTFKCYGVSPTKLNDLCFTNV
PF2 (30 aa):
(SEQ ID NO: 2)
GVSPTKLNDLCFTNVYADSFVIRGDEVRQI
PF3 (30 aa):
(SEQ ID NO: 3)
YADSFVIRGDEVRQIAPGQTGKIADYNYKL
PF4 (30 aa):
(SEQ ID NO: 4)
APGQTGKIADYNYKLPDDFTGCVIAWNSNN
PF5 (30 aa):
(SEQ ID NO: 5)
PDDFTGCVIAWNSNNLDSKVGGNYNYLYRL
PF6 (30 aa):
(SEQ ID NO: 6)
LDSKVGGNYNYLYRLFRKSNLKPFERDIST
PF7 (30 aa):
(SEQ ID NO: 7)
FRKSNLKPFERDISTEIYQAGSTPCNGVEG
PF8 (30 aa):
(SEQ ID NO: 8)
EIYQAGSTPCNGVEGFNCYFPLQSYGFQPT
PF9 (31 aa):
(SEQ ID NO: 9)
FNCYFPLQSYGFQPTNGVGYQPYRVVVLSFE
PF10 (36 aa):
(SEQ ID NO: 10)
DISGINASVVNIQKEIDRLNVAKNLNESLIDLQELG
PF11 (30 aa):
(SEQ ID NO: 11)
EIDRLNEVAKNLNESLIDLQELGKYEQYIKW
PF12 (30 aa):
(SEQ ID NO: 12)
IDLQELGKYEQYIKWPWYIWLGFIAGLIAIV

In such an embodiment, the native protein sequence is the S protein of SARS-Cov-2 and has an amino acid sequence (Uniprot accession number PODTC2) as follows:

(SEQ ID NO: 13)
MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNWVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKFNGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT

In some embodiments, the portion of the native protein sequence is the S1 subunit of the S protein of SARS-CoV-2 having the following sequence:

(SEQ ID NO: 14)
SQCVNLTTRTQLPPAYTNSFTRGVYYPDKVFRSSVLHSTQ
DLFLPFFSNVTWFHAIHVSGTNGTKRFDNPVLPFNDGVYF
ASTEKSNIIRGWIFGTTLDSKTQSLLIVNNATNVVIKVCE
FQFCNDPFLGVYYHKNNKSWMESEFRVYSSANNCTFEYVS
QPFLMDLEGKQGNFKNLREFVFKNIDGYFKIYSKHTPINL
VRDLPQGFSALEPLVDLPIGINITRFQTLLALHRSYLTPG
DSSSGWTAGAAAYYVGYLQPRTFLLKYNENGTITDAVDCA
LDPLSETKCTLKSFTVEKGIYQTSNFRVQPTESIVRFPNI
TNLCPFGEVFNATRFASVYAWNRKRISNCVADYSVLYNSA
SFSTFKCYGVSPTKLNDLCFTNVYADSFVIRGDEVRQIAP
GQTGKIADYNYKLPDDFTGCVIAWNSNNLDSKVGGNYNYL
YRLFRKSNLKPFERDISTEIYQAGSTPCNGVEGFNCYFPL
QSYGFQPTNGVGYQPYRVVVLSFELLHAPATVCGPKKSTN
LVKNKCVNFNFNGLTGTGVLTESNKKFLPFQQFGRDIADT
TDAVRDPQTLEILDITPCSFGGVSVITPGTNTSNQVAVLY
QDVNCTEVPVAIHADQLTPTWRVYSTGSNVFQTRAGCLIG
AEHVNNSYECDIPIGAGICASYQTQTNSPRRAR

In some embodiments, the portion of the native protein sequence is the RBD and has the amino acid sequence as follows, or a variant thereof:

(SEQ ID NO: 15)
CPFGEVFNATRFASVYAWNRKRISNCVADYSVLYNSASFS
TFKCYGVSPTKLNDLCFTNVYADSFVIRGDEVRQIAPGQT
GKIADYNYKLPDDFTGCVIAWNSNNLDSKVGGNYNYLYRL
FRKSNLKPFERDISTEIYQAGSTPCNGVEGFNCYFPLQSY
GFQPTNGVGYQPYRVVVLSFE
or
(SEQ ID NO: 16)
RVQPTESIVRFPNITNLCPFGEVFNATRFASVYAWNRKRI
SNCVADYSVLYNSASFSTFKCYGVSPTKLNDLCFTNVYAD
SFVIRGDEVRQIAPGQTGKIADYNYKLPDDFTGCVIAWNS
NNLDSKVGGNYNYLYRLFRKSNLKPFERDISTEIYQAGST
PCNGVEGFNCYFPLQSYGFQPTNGVGYQPYRVVVLSFELL
HAPATVCGPKK
or
(SEQ ID NO: 50)
ITNLCPFGEVFNATRFASVYAWNRKRISNCVADYSVLYNS
ASFSTFKCYGVSPTKLNDLCFTNVYADSFVIRGDEVRQIA
PGQTGKIADYNYKLPDDFTGCVIAWNSNNLDSKVGGNYNY
LYRLFRKSNLKPFERDISTEIYQAGSTPCNGVEGFNCYFP
LQSYGFQPTNGVGYQPYRVVVLSFELLHAPATV

In some embodiments, the portion of the native protein sequence is the RBM having the amino acid sequence as follows, or a variant thereof:

(SEQ ID NO: 17)
SNNLDSKVGGNYNYLYRLFRKSNLKPFERDISTEIYQAGS
TPCNGVEGFNCYFPLQSYGFQPTNGVGYQ
or
(SEQ ID NO: 51)
QTGKIADYNYKLPDDFTGCVIAWNSNNLDSKVGGNYNYLY
RLFRKSNLKPFERDISTEIYQAGSTPCNGVEGFNCYFPLQ
SYGFQPTNGVGYQ

SEQ ID NOs: 10 to 12 comprise sequences derived from the S2 subunit of the S protein of SARS-CoV-2. In some embodiments, the portion of the native protein sequence is the S2 subunit having the amino acid sequence as follows, or a variant thereof:

(SEQ ID NO: 18)
SVASQSIIAYTMSLGAENSVAYSNNSIAIPTNFTISVTTE
ILPVSMTKTSVDCTMYICGDSTECSNLLLQYGSFCTQLNR
ALTGIAVEQDKNTQEVFAQVKQIYKTPPIKDFGGFNFSQI
LPDPSKPSKRSFIEDLLFNKVTLADAGFIKQYGDCLGDIA
ARDLICAQKFNGLTVLPPLLTDEMIAQYTSALLAGTITSG
WTFGAGAALQIPFAMQMAYRFNGIGVTQNVLYENQKLIAN
QFNSAIGKIQDSLSSTASALGKLQDVVNQNAQALNTLVKQ
LSSNFGAISSVLNDILSRLDKVEAEVQIDRLITGRLQSLQ
TYVTQQLIRAAEIRASANLAATKMSECVLGQSKRVDFCGK
GYHLMSFPQSAPHGVVFLHVTYVPAQEKNFTTAPAICHDG
KAHFPREGVFVSNGTHWFVTQRNFYEPQIITTDNTFVSGN
CDVVIGIVNNTVYDPLQPELDSFKEELDKYFKNHTSPDVD
LGDISGINASVVNIQKEIDRLNEVAKNLNESLIDLQELGK
YEQYIKWPWYIWLGFIAGLIAIVMVTIMLCCMTSCCSCLK
GCCSCGSCCKFDEDDSEPVLKGVKLHYT

SEQ ID NO: 18 consists of residues 686-1273 of SEQ ID NO: 13.

SEQ ID NOs: 10 to 12 comprise sequences derived, at least in part, from the HR2 region of the S2 subunit of the S protein of SARS-CoV-2. In some embodiments, the portion of the native protein sequence is the HR2 region having the amino acid sequence as follows, or a variant thereof:

(SEQ ID NO: 19)
HTSPDVDLGDISGINASVVNIQKEIDRLNEVAKNLNESLI
DLQELGKYEQYIK

In some embodiments, the polypeptide comprises peptide fragments comprising sequences derived, at least in part, from the HR1 region of the S2 subunit of the S protein of SARS-CoV-2. In some embodiments, the portion of the native protein sequence is the HR1 region having the amino acid sequence as follows, or a variant thereof:

(SEQ ID NO: 20)
GVTQNVLYENQKLIANQFNSAIGKIQDSLSSTASALGKLQ
DVVNQNAQALNTLVKQLSSNFGAISSVLNDILSRLDKVE

SEQ ID NO: 20 consists of residues 910-988 of SEQ ID NO: 13. In some embodiments the native protein sequence is survivin, chosen from any one of the following survivin isoforms:

(142 aa)
(SEQ ID NO: 21)
MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIAKETNNK
KKEFEETAKK VRRAIEQLAA MD

This relates to survivin isoform 1 (uniprot identifier 015392-1), but in some embodiments the sequences could be derived from or variants of one or more of survivin isoform 2 (uniprot identifier 015392-2, SEQ ID NO: 22), 3 (uniprot identifier 015392-3, SEQ ID NO: 23), 4 (uniprot identifier 015392-4, SEQ ID NO: 24), 5 (uniprot identifier 015392-5, SEQ ID NO: 25), 6 (uniprot identifier 015392-6, SEQ ID NO: 26), or 7 (uniprot identifier 015392-7, SEQ ID NO: 27).

(165 aa)
(SEQ ID NO: 22)
MGAPTLPPAW QPFLKDHRIS TFKNWPFLEG CACTPERMAE
AGFIHCPTEN EPDLAQCFFC FKELEGWEPD DDPIGPGTVA
YACNTSTLGG RGGRITREEH KKHSSGCAFL SVKKQFEELT
LGEFLKLDRE RAKNKIAKET NNKKKEFEET AKKVRRAIEQ
LAAMD
(137 aa)
(SEQ ID NO: 23)
MGAPTLPPAW QPFLKDHRIS TFKNWPFLEG CACTPERMAE
AGFIHCPTEN EPDLAQCFFC FKELEGWEPD DDPMQRKPTI
RRKNLRKLRR KCAVPSSSWL PWIEASGRSC LVPEWLHHFQ
GLFPGATSLP VGPLAMS
(120 aa)
(SEQ ID NO: 24)
MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLDRERAK NKIERALLAE
(117 aa)
(SEQ ID NO: 25)
MGAPTLPPAW QPFLKDHRIS TFKNWPFLEG CACTPERMAE
AGFIHCPTEN EPDLAQCFFC FKELEGWEPD DDPIEEHKKH
SSGCAFLSVK KQFEELTLGE FLKLVRETLP PPRSFIR
(78 aa)
(SEQ ID NO: 26)
MGAPTLPPAW QPFLKDHRIS TFKNWPFLEG CACTPERMAE
AGFIHCPTEN EPDLAQCFFC FKELEGWEPD DDPMRELC
(74 aa)
(SEQ ID NO: 27)
MGAPTLPPAW QPFLKDHRIS TFKNWPFLEG CACTPERMAE
AGFIHCPTEN EPDLAQCFFC FKELEGWEPD DDPM

The skilled reader will understand that a nucleic acid sequence (DNA or RNA, or a mix of both) can be provided for each of the above-mentioned peptides, and that this would be routine for the person skilled in the art to derive. For example, the DNA sequence encoding SEQ ID NO: 21 is given below.

(426 bp)
(SEQ ID NO: 28)
ATGGGCGCCCCCACCCTGCCCCCCGCCTGGCAGCCCTTCCT
GAAGGACCACAGGATCAGCACCTTCAAGAACTGGCCCTTC
CTGGAGGGCTGCGCCTGCACCCCCGAGAGGATGGCCGAGG
CCGGCTTCATCCACTGCCCCACCGAGAACGAGCCCGACCT
GGCCCAGTGCTTCTTCTGCTTCAAGGAGCTGGAGGGCTGG
GAGCCCGACGACGACCCCATCGAGGAGCACAAGAAGCACA
GCAGCGGCTGCGCCTTCCTGAGCGTGAAGAAGCAGTTCGA
GGAGCTGACCCTGGGCGAGTTCCTGAAGCTGGACAGGGAG
AGGGCCAAGAACAAGATCGCCAAGGAGACCAACAACAAGA
AGAAGGAGTTCGAGGAGACCGCCAAGAAGGTGAGGAGGGC
CATCGAGCAGCTGGCCGCCATGGAC

In some embodiments, at least one of the two or more peptide fragments comprises a sequence with at least 90% identity to a sequence selected from the group:

(30 aa)
(SEQ ID NO: 29)
MGAPTLPPAWQPFLKDHRISTFKNWPFLEG
(30 aa)
(SEQ ID NO: 30)
DHRISTFKNWPFLEGCACTPERMAEAGFIH
(28 aa)
(SEQ ID NO: 31)
ACTPERMAEAGFIHCPTENEPDLAQCFF
(29 aa)
(SEQ ID NO: 32)
PTENEPDLAQCFFCFKELEGWEPDDDPIE
(30 aa)
(SEQ ID NO: 33)
FKELEGWEPDDDPIEEHKKHSSGCAFLSVK
(28 aa)
(SEQ ID NO: 34)
EHKKHSSGCAFLSVKKQFEELTLGEFLK
(29 aa)
(SEQ ID NO: 35)
QFEELTLGEFLKLDRERAKNKIAKETNNK
(30 aa)
(SEQ ID NO: 36)
RERAKNKIAKETNNKKKEFEETAEKVRRAI
(21 aa)
(SEQ ID NO: 37)
KEFEETAEKVRRAIEQLAAMD

and the polypeptide elicits an immune response or is immunostimulatory.

In some embodiments, the two or more peptide fragments comprise a sequence with at least 90% identity to SEQ ID NO 32 and/or SEQ ID NO 33 and the polypeptide elicits an immune response, optionally a T-cell response.

In some embodiments, the native protein sequence is an E6 or E7 protein of a Human papillomavirus (HPV).

In some embodiments, the native protein sequence is:

(158 aa)
(SEQ ID NO: 38)
MHQKRTAMFQDPQERPRKLPQLCTELQTTIHDIILE
CVYCKQQLLRREVYDFAFRDLCIVYRDGNPYAVCDK
CLKFYSKISEYRHYCYSLYGTTLEQQYNKPLCDLLI
RCINCQKPLCPEEKQRHLDKKQRFHNIRGRWTGRCM
SCCRSSRTRRETQL
or
(98 aa)
(SEQ ID NO: 39)
MHGDTPTLHEYMLDLQPETTDLYCYEQLNDSSEEED
EIDGPAGQAEPDRAHYNIVTFCCKCDSTLRLCVQST
HVDIRTLEDLLMGTLGIVCPICSQKP

SEQ ID NO: 38 is the E6 peptide of Human papilloma virus 16 (Uniprot identifier: P03126). SEQ ID NO: 39 is the E7 peptide of Human papilloma virus 16 (Uniprot identifier: P03129).

In some embodiments, at least one of the two or more peptide fragments comprises a sequence with at least 90% identity to a sequence selected from the group:

(35 aa)
(SEQ ID NO: 40)
MHGDTPTLHEYMLDLQPETTDLYCYEQLNDSSEEE
(35 aa)
(SEQ ID NO: 41)
EQLNDSSEEEDEIDGPAGQAEPDRAHYNIVTFCCK
(35 aa)
(SEQ ID NO: 43)
HYNIVTFCCKCDSTLRLCVQSTHVDIRTLEDLLMG
(23 aa)
(SEQ ID NO: 42)
IRTLEDLLMGTLGIVCPICSQKP

In a further aspect, the invention provides one or more polynucleotides encoding a native protein sequence or portion thereof and/or one or more polynucleotides encoding a polypeptide comprising two or more peptide fragments, wherein a first peptide fragment comprises a first sequence derived from a native protein sequence and wherein a second peptide fragment comprises a second sequence derived from the native protein sequence, further comprising one or more protease cleavage site sequences located between each of the two or more peptide fragments. In other words, the polypeptide and/or native protein of the first aspect can be provided as one or more polynucleotides encoding said polypeptide and/or native protein. As such, any of the embodiments of the first aspect are equally applicable to this aspect, and it would be routine for the skilled person to derive the encoding sequence of the polynucleotide required for any of SEQ ID NOs: 1 to 27 and 29 to 43, as illustrated above with SEQ ID NO: 28.

In a further aspect of the invention is provided a method for the immunization and/or treatment of a subject comprising administering, to the subject, the formulation of any one of the previous aspects. In some embodiments, the polypeptide and the native protein sequence or portion thereof are administered to a subject simultaneously, separately, or sequentially.

A further aspect of the invention provides a composition for use in the immunization and/or treatment of a subject, wherein the composition comprises the polypeptide and the native protein sequence or fragment thereof of the formulation of the previous aspects, and wherein the polypeptide with the native protein sequence or portion thereof, or the one or more polynucleotides encoding the native protein sequence or portion thereof and/or the polypeptide are co-administered.

A further aspect of the invention provides a method of manufacturing a vaccine comprising expressing one or more polynucleotides encoding the native protein sequence or portion thereof and the polypeptide as described in any previous aspect, in one or more cells in vitro, and purifying the native protein sequence or portion thereof and the polypeptide. In some embodiments, the purified native protein sequence or portion thereof and the polypeptide are combined into a single formulation.

A further aspect of the invention provides a kit for the immunization and/or treatment of a subject, comprising the native protein sequence or portion thereof of any one of the aforementioned aspects, or one or more polynucleotides encoding the native protein sequence or portion thereof, and the polypeptide of the aforementioned aspects, or one or more polynucleotides encoding the polypeptide. In some embodiments the kit further comprises a pharmaceutically acceptable carrier.

A further aspect of the invention provides a method for the immunization and/or treatment of a subject comprising: administering the native protein sequence or portion thereof of any of the preceding aspects, or one or more polynucleotides encoding the native protein sequence or portion thereof, and administering the polypeptide of any of the preceding aspects, or one or more polynucleotides encoding the polypeptide.

In some embodiments, wherein the native protein sequence or portion thereof, or one or more polynucleotides encoding the native protein sequence or portion thereof are administered simultaneously, sequentially, or separately to the polypeptide or one or more polynucleotides encoding the polypeptide.

It will be understood that such a method also provides a composition for use according to the method, said composition comprising the polypeptide, the native protein or portion thereof, and/or the one or more polynucleotides encoding the polypeptide, and/or the one or more polynucleotides encoding the native protein or portion thereof, as described above.

It will be understood by the skilled reader that in certain embodiments the native protein sequence is not from a coronavirus, in particular it is not the RBD, RBM, and/or S1 and/or S2 peptide sequences. It will be appreciated that whilst certain embodiments may relate to the coronavirus, HPV, and/or survivin, the present invention is broadly applicable across any number of native protein sequences for which it is desirable to raise an immunogenic response. The skilled person, having understood the present disclosure, would be able to derive a suitable ROP, and combine it with the native protein or portion thereof upon which it is based in order to produce a highly immunogenic vaccine formulation according to the present invention.

### Example 1—Combining an ROP Raised Against SARS-CoV-2 Proteins with a Portion of the Native Spike Protein Sequence

A polypeptide vaccine (‘ROP-COVS’) was designed towards the SARS-CoV-2 protein domains which are most actively involved in viral entry into a host cell. It has the following amino acid sequence:

(SEQ ID NO: 44)
MHHHHHHSVLYNSASFSTFKCYGVSPTKLND
LCFTNVLRMKGVSPTKLNDLCFTNVYADSFV
IRGDEVRQILRMKYADSFVIRGDEVRQIAPG
QTGKIADYNYKLLRMKAPGQTGKIADYNYKL
PDDFTGCVIAWNSNNLRMKPDDFTGCVIAWN
SNNLDSKVGGNYNYLYRLLRMKLDSKVGGNY
NYLYRLFRKSNLKPFERDISTLRMKERKSNL
KPFERDISTEIYQAGSTPCNGVEGLRMKEIY
QAGSTPCNGVEGENCYFPLQSYGFQPTLRMK
ENCYFPLQSYGFQPTNGVGYQPYRVVVLSFE
LRMKDISGINASVVNIQKEIDRLNEVAKNLN
ESLIDLQELGLRMKEIDRLNEVAKNLNESLI
DLQELGKYEQYIKWLRMKIDLQELGKYEQYI
KWPWYIWLGFIAGLIAIV

This ROP-COVS is a recombinant polypeptide comprising 12 peptide fragments (‘PF’s), each linked to the next via a LRMK cleavage sequence of cathepsin S, such that the PFs can be liberated intracellularly upon digestion by cathepsin S. Each PF is numbered 1 to 12 according to sequential amino acid position within the ROP, with PF1 being the OSP most proximate to the N-terminus and PF12 the most proximate to the C-terminus. The sequences of the PFs are as follows:

PF1 (30 aa):
(SEQ ID NO: 1)
SVLYNSASFSTFKCYGVSPTKLNDLCFTNV
PF2 (30 aa):
(SEQ ID NO: 2)
GVSPTKLNDLCFTNVYADSFVIRGDEVRQI
PF3 (30 aa):
(SEQ ID NO: 3)
YADSFVIRGDEVRQIAPGQTGKIADYNYKL
PF4 (30 aa):
(SEQ ID NO: 4)
APGQTGKIADYNYKLPDDFTGCVIAWNSNN
PF5 (30 aa):
(SEQ ID NO: 5)
PDDFTGCVIAWNSNNLDSKVGGNYNYLYRL
PF6 (30 aa):
(SEQ ID NO: 6)
LDSKVGGNYNYLYRLERKSNLKPFERDIST
PF7 (30 aa):
(SEQ ID NO: 7)
FRKSNLKPFERDISTEIYQAGSTPCNGVEG
PF8 (30 aa):
(SEQ ID NO: 8)
EIYQAGSTPCNGVEGENCYFPLQSYGFQPT
PF9 (31 aa):
(SEQ ID NO: 9)
FNCYFPLQSYGFQPTNGVGYQPYRVVVLSFE
PF10 (36 aa):
(SEQ ID NO: 10)
DISGINASVVNIQKEIDRLNVAKNLNESLIDLQELG
PF11 (30 aa):
(SEQ ID NO: 11)
EIDRLNEVAKNLNESLIDLQELGKYEQYIKW
PF12 (30 aa):
(SEQ ID NO: 12)
IDLQELGKYEQYIKWPWYIWLGFIAGLIAIV

Each PF shares a portion of its sequence (aka ‘overlaps’) with at least one other. For example, amino acids 1 to 15 of PF2 comprise amino acids 16 to 30 of PF1 i.e. a so-called overlap; amino acids 1 to 15 of PF3 comprise amino acids 16 to 30 of PF2 i.e. another so-called overlap.

### 1. Gene Sequence Design

PFs 1 to 9 were selected to tile the SARS-CoV-2 S1 receptor binding domain (‘RBD’) (SEQ ID NO: 15 or 16) and to comprise a number of whole or partial antibody and T cell epitopes of the RBD. PFs 10 to 12 were selected to tile the C-terminal end of the SARS-CoV-2 S2 HR2 region (SEQ ID NO: 19) and the proximal region of S2 (amino acids 483 to 543 of SEQ ID NO: 18), and to comprise whole or partial antibody and T cell epitopes thereof. Each PF, or ‘peptide fragment’, is linked to the next by a LRMK sequence. The resulting designed ROP-COVS is illustrated schematically in FIG. 4. A N-terminal His 6 tag was added for purification of the ROP-COVS.

The E. coli codon-optimized gene sequence encoding the resultant His-tagged ROP-COVS protein is presented as SEQ ID NO: 45.

(SEQ ID NO: 45)
CATATGCATCACCACCATCATCATAGCGTCCTGTACAACA
GCGCGAGCTTTTCCACCTTCAAATGCTACGGCGTTAGTCC
GACCAAACTGAACGATCTGTGCTTCACCAACGTCCTGCGT
ATGAAAGGCGTTAGTCCGACCAAACTGAACGATCTGTGCT
TCACCAACGTCTACGCAGATAGCTTCGTAATTCGCGGCGA
CGAAGTTCGTCAAATCCTGCGCATGAAATACGCCGATAGC
TTCGTAATTCGCGGCGACGAAGTTCGTCAAATTGCACCGG
GTCAAACCGGTAAAATTGCGGATTACAACTACAAACTGCT
GCGCATGAAAGCACCGGGTCAGACCGGCAAAATTGCGGAT
TACAACTACAAACTGCCGGACGATTTCACCGGTTGCGTTA
TTGCCTGGAACAGCAACAACCTGCGTATGAAACCGGACGA
TTTCACCGGTTGCGTTATTGCCTGGAACAGCAACAACCTG
GATAGCAAAGTCGGCGGCAACTACAACTACCTGTATCGTC
TGCTGCGCATGAAACTGGATAGCAAAGTGGGCGGCAACTA
TAACTACCTGTATCGTCTGTTCCGCAAAAGCAACCTGAAA
CCGTTCGAACGCGATATTTCCACCCTGCGCATGAAATTCC
GCAAAAGCAACCTGAAACCGTTCGAGCGCGATATCAGCAC
CGAAATTTATCAGGCCGGTTCTACCCCGTGTAACGGCGTT
GAAGGTCTGCGCATGAAAGAAATCTACCAGGCAGGTTCTA
CCCCGTGTAACGGCGTTGAAGGTTTCAACTGCTACTTTCC
GCTGCAGAGTTACGGTTTTCAGCCGACCCTGCGCATGAAA
TTCAACTGCTACTTTCCGCTGCAGAGCTACGGTTTTCAAC
CGACCAACGGCGTAGGTTATCAACCGTATCGCGTTGTCGT
TCTGAGCTTCGAACTGCGCATGAAAGACATCAGCGGTATT
AACGCGAGCGTCGTCAACATCCAGAAAGAGATCGACCGTC
TGAACGAAGTCGCGAAAAACCTGAACGAGAGCCTGATTGA
TCTGCAAGAACTGGGTCTGCGCATGAAAGAAATCGACCGT
CTGAACGAAGTCGCGAAAAACCTGAACGAAAGCCTGATCG
ATCTGCAGGAACTGGGCAAATACGAGCAGTACATCAAATG
GCTGCGCATGAAAATCGATCTGCAGGAACTGGGCAAATAC
GAGCAGTACATCAAATGGCCGTGGTACATCTGGCTGGGTT
TTATTGCCGGTCTGATCGCGATTGTGTAATGAAAGCTT

This gene sequence was cloned through clonal amplification in DH5α E. coli, identified via colony PCR and inserted into a pET30a vector (forming plasmid Y0028023-1, FIG. 1).

### 2. Protein Production

### 2.1 Expression

Plasmid Y0028023-1 was transformed into BL21 (DE3) E. coli. Electrophoretic analysis confirmed that the ROP-COVS gene inserted successfully (FIG. 2). Flasks (250 mL) containing 50 mL of LB medium (containing 50 μg/ml kanamycin sulfate) were used for cultivation. The strain was inoculated at a proportion of 1:500. The bacteria were incubated with rotary shaking (150 rpm) at 37° C. overnight. The cell culture was transferred into 1.2 L 2YT medium (containing 50 μg/ml kanamycin sulfate) at a proportion of 1:100. Once the OD600 value reached 0.8, IPTG was added to a final concentration of 0.2 mM to induce the expression of ROP-COVS. The bacteria were incubated with rotary shaking of 200 rpm at 37° C.

Bacteria were harvested by centrifugation at 4500 rpm for 30 min. SDS-PAGE was used to analyze the induction rate and demonstrated successful induction after a 4-hour incubation (FIG. 3A).

### 2.2 Collection of Inclusion Bodies

The harvested wet bacteria were resuspended and washed once with 0.9% NaCl, with a washing ratio of 10 ml/g and centrifugal conditions of 4500 rpm, 4° C., 30 min. After washing, the wet bacteria were dissolved with lysis buffer (20 mM Tris-HCl, 300 mM NaCl, 20 mM Imidazole, 1% Triton X-100, 1 mM DTT, 1 mM PMSF, pH 8.0) in 10 ml/g, and the pellets were lysed by sonication for 60 cycles (3 s on and 5 s off).

After lysis, the soluble and insoluble fractions were analysed by SDS-PAGE, which indicated that the target protein ROP-COVS was expressed as inclusion bodies in cells. The inclusion bodies were collected by centrifugation at 9500 rpm for 30 min. The supernatant was discarded. The inclusion bodies were then washed twice with washing buffer 1 (20 mM Tris-HCl, 300 mM NaCl, 1% Triton X-100, 2 mM EDTA, 5 mM DTT, pH 8.0) and once with washing buffer 2 (20 mM Tris-HCl, pH8.0).

### 2.3 Purification

The inclusion bodies were dissolved in Buffer A (20 mM Tris-HCl, 300 mM NaCl, 8 M Urea, pH 8.0) and magnetically mixed over night at 4° C. The suspension was subjected to centrifugation (18000 rpm, 30 min, 4° C.) to remove the undissolved fractions. The supernatant was loaded into the Ni-NTA column (Smart-Lifesciences) pre-equilibrated with Buffer A. Fractions containing target protein were eluted with a 0-300 mM Imidazole gradient in 20 mM Tris-HCl buffer containing 300 mM NaCl (pH 8.0). SDS-PAGE was used to analyze the result of purification (FIG. 3B).

### 2.4 Refolding

Fractions with over 95% purity (those in Lanes 6-8 of FIG. 3B) were collected. Dialysis was used for refolding: the fractions were first dialyzed to refolding buffer 1 (1×PBS, 4 mM GSH, 0.4 mM GSSG, 0.4 M L-Arginine, 1 M Urea, 5% Glycerol, 0.5% Sarkosyl, pH 7.4) and then to dialyzed to refolding buffer 2 (1×PBS, 5% glycerol). SDS-PAGE was used to analyze the refolding result (FIG. 3C).

### 2.5 Production of SARS-CoV-2 RBM

In some embodiments, RBM is co-administered with ROP-COVS. SARS-CoV-2 RBM (SEQ ID NO: 17) was expressed from E. coli according to standard procedures and purified by affinity chromatography according to standard procedures.

### 3. Vaccination and Demonstration of Immune Response

### 3.1 Preparation of BALB/c Mice

40 mice (SPF grade, 5-6 weeks, female) were bought from Changzhou Cavens Co., Ltd. To allow them to adapt to the new environment, the mice were fed for one week before vaccination.

### 3.2 Vaccination

RBM is co-administered with ROP-COVS. The mice were divided into three groups and vaccinated according to Table 2, below. The mice were vaccinated on day 0, day 14 and day 21. Each mouse was injected subcutaneously with 100 μl total mixture of Antigens (or S protein as a control (SEQ ID NO: 13)) and MPL. The dose of MPL was followed as per the instruction. At day 24, 3 days after the final vaccination, all mice were sacrificed.

TABLE 1
ROP-COV vaccination regime 2

| | | Vaccination (V) Group 1 | | | V Group 2 | | | Control (C) Group | | |
|---|---|---|---|---|---|---|---|---|---|---|
| Dose | Day | No. | Antigen | Adjuvant | No. | Antigen | Adjvnt | No. | Antigen | Adjvnt |
| 1st | 0 | 5 | 50 μg | MPL | 5 | 50 μg | MPL | 5 | 50 μg S | MPL |
| | | | ROP-COVS | | | ROP-COVS + | | | protein | |
| | | | | | | 50 μg RBM | | | | |
| 2nd | 14 | | 25 μg | MPL | | 25 μg | MPL | | 50 μg S | MPL |
| | | | ROP-COVS | | | ROP-COVS + | | | protein | |
| | | | | | | 25 μg RBM | | | | |
| 3rd | 21 | | 25 μg | MPL | | 25 μg | MPL | | 50 μg S | MPL |
| | | | ROP-COVS | | | ROP-COVS + | | | protein | |
| | | | | | | 25 μg RBM | | | | |

### 3.3 Surrogate Neutralization Assay (ELISA)

Mouse sera was extracted and separated as per 3.3 from mice vaccinated according to the above protocols. An ELISA-based surrogate neutralization assay based on Ig competition with the hACE2-RBD binding interaction was carried out according to the following protocol:
1. Plate coated with 20 μg/ml RBD, 100 μl/well, incubated at 4° C. overnight.
2. Plate washed with PBST. Plate blocked with 2.5% BSA, 200 μl/well, incubated at 37° C. for 1 h.
3. Plate washed with PBST. The mice sera were diluted to 1:100, 1:200, 1:400, 1:800 and 1:1600. The sera with different dilution were added into the plate, 50 μl/well. 20 μg/ml ACE2-hFc were then added into the plate, 50 μl/well. The plate was incubated at 37° C. for 30 min.
4. Plate washed with PBST. 100 μl/well of anti-hFc antibodies (HRP conjugated) were added. The plate was incubated at room temperature for 30 min.
5. Plate washed with PBST. 100 μl/well TMB substrate added, and allowed to stand for 5-10 min.
6. 50 μl/well of 15% H2SO4 was added into the plate. The OD value was read at 450 nm.

Alternative ELISA-based surrogate neutralization assays are also suitable, for example as described in Tan, C. W., et al. (2020).

Results are shown in FIG. 5. Vaccination with ROP-COVS stimulates higher neutralizing antibody titres than vaccination with S protein (shown by lower absorbance). Vaccination with both ROP-COVS and RBM produces the highest neutralizing antibody titres. The data show that the combination of a portion of the native sequence and an ROP based upon said native sequence produce a greater inhibition of the hACE2-RBD binding interaction than the ROP alone, or the native protein alone. This means that the antibodies produced by the combined approach are either higher affinity or more are produced, In this case, the full length spike protein was used as a control. It is surprising that the RBM plus ROP produces a greater immune response than the spike protein alone, as the spike protein contains a longer amino acid sequence with potentially more epitopes therein to stimulate an immune response. However, the combination of the highly immunogenic ROP structure plus a portion of the Spike protein (the RBM) appears to produce a much greater antibody titre than either alone.

This surrogate neutralization assay was repeated using IgG purified from mice sera according to standard protocols. Results are shown in FIG. 6. At concentrations of 100 μg/ml and below, neutralizing titres in response to ROP-COVS are higher than to S protein. Vaccination with a combination of ROP-COVS and RBM again stimulates highest neutralizing antibody titres at all IgG concentrations.

### 3.4 Neutralization Assay

Mice are vaccinated according to Regime 1 and/or 2 and sera extracted and separated as per 3.3. Neutralization assays are performed with pseudotyped or chimeric SARS-CoV-2 virus particles according to standard protocols for example as described in Nie, J., et al. or Schmidt F, et al. More preferably, neutralization assays are carried out using replication-competent SARS-CoV-2 at BSL-3 using standard protocol as described in Amanat, F., et al.

Results demonstrate that antibodies produced by mice vaccinated with ROP-COVS and with a combination of ROP-COVS+RBM block viral entry and/or replication. Results demonstrate that the combination of ROP-COVS+RBM is more potent for generation of neutralizing antibodies than ROP-COVS alone.

### 3.5 ELISPOT Analysis of T Cell Response

Spleens are extracted from sacrificed mice (having been vaccinated according to Regime 1 and/or 2) and are strained through a mesh, loaded to murine splenocyte separation medium (Solarbio), and centrifuged at 1000 g for 22 minutes before transferring the layered lymphocytes to a new tube with cell culture medium. The cells are washed twice by RPMI 1640. 2.5×10 5 splenocytes per well will be used for stimulation in ELISPOT assays. CD4⁺ or CD8⁺ T cells are purified by negative or positive selection using microbeads kit (Miltenyi, Germany) as per the manufacturer's instructions. Assays are performed using ELISPOT kits (Mabtech, Sweden).

Briefly, splenocytes are restimulated overnight with 5 μg/well SARS-CoV-2 S protein or ROP-COVS in anti-5 IFN-γ-Ab precoated plates (Millipore). Cells are discarded, and biotinylated anti-IFN-γ antibody are added for two hours at room temperature, followed by another one hour of incubation at room temperature with alkaline phosphatase (ALP) conjugated streptavidin. After color develops, the reaction is stopped by washing plates with tap water and plates are air-dried. Spots will be counted with an ELISPOT reader (CTL). Results demonstrate that ROP-COVS can stimulate pronounced CD4+ and CD8+ T cell responses.

### 3.6 Pre-Clinical Trials

In vivo pre-clinical trials can be conducted following standard protocols (see e.g. Munoz-Fontela, C., et al.). Neutralizing antibody titres and ELISpot assay PMBC T cell responses are measured. Results demonstrate that vaccination with ROP-COVS alone or in combination with RBM generates protective anti-SARS-CoV-2 immune responses.

### Example 2—Combining a Survivin-ROP with the Native Peptide Survivin

### Materials and Methods

In order to validate the approach combining the native protein sequence with the polypeptide as described above, a mouse model using a mouse survivin and a recombinant overlapping peptide (ROP) with the ability to raise an immune response against mouse survivin was used. It will be understood that the below sequences are included with a His-tag, but this is optional and may be removed or replaced with another tag.

### 1. Sequences

The mouse survivin sequence used herein is as follows:

Mouse Survivin:

(SEQ ID NO: 46)
MHHHHHHGAP ALPQIWQLYL KNYRIATFKN WPFLEDCACT
PERMAEAGFI HCPTENEPDL AQCFFCFKEL EGWEPDDNPI
EEHRKHSPGC AFLTVKKQME ELTVSEFLKL DRQRAKNKIA
KETNNKQKEF EETAKTTRQS IEQLAA

The sequence of the ROP is as follows:

Mouse ROP-Survivin:

(SEQ ID NO: 47)
MHHHHHHGAPALPQIWQLYLKNYRIATFKNWPFLEDLRMK
NYRIATFKNWPFLEDCACTPERMAEAGFIHLRMKCACTPE
RMAEAGFIHCPTENEPDLAQCFFCLRMKCPTENEPDLAQC
FFCFKELEGWEPDDNPIELRMKFKELEGWEPDDNPIEEHR
KHSPGCAFLTVKLRMKEHRKHSPGCAFLTVKKQMEELTVS
EFLKLDLRMKKQMEELTVSEFLKLDRQRAKNKIAKETNNK
LRMKRQRAKNKIAKETNNKQKEFEETAKTTRQSIEQLAA

### 2. Animals

Female C57BL/6 mice were purchased from Changzhou Kavins Experimental Animal Co. LTD. The animals were specific pathogen free and approximately 6-7 weeks old upon arrival. Upon receipt the animals were unpacked and placed in cages. A health inspection was performed on each animal to include evaluation of the coat, extremities and orifices. Each animal was also examined for any abnormal signs in posture or movement. The animals were housed in clear polycarbonate plastic cages (260 mm×160 mm×120 mm); 2-5 animals per cage. The bedding material was corn-cob bedding (irradiated, Shandong Goodway Biotechnology Co., Ltd., China) that was changed once a week. The room was supplied with H EPA filtered air at the rate of 15-25 air changes per hour. The temperature was maintained at 20-26° C. (68-79° F.). Illumination was fluorescent light for 12-hour light (08:00-20:00) and 12-hour dark. Animals had ad libitum access to rodent food (Shuck Beta Co., Ltd., China). Water, from the municipal water supply, was filtered by reverse osmosis or high-pressure sterilizer.

### 3. Expression/Purification

The expression of N-terminal His-tagged ROP-Survivin or survivin protein was induced by 0.2 mM IPTG when the OD600 reached 0.5-0.8. The induction was performed at 15° C. for 16 hours.

For preparing bacterial lysates, the bacteria were suspended in 20 mM PB (pH7.2, containing 300 mM NaCl, 20 mM Imidazole, 1% Triton X-100, 1 mM DTT and 1 mM PMSF) and sonicated. Inclusion body (IB) was washed by 20 mM PB (pH7.2, containing 300 mM NaCl, 1% Triton X-100, 2 mM EDTA and 5 mM DTT). Finally, the cleaned IB was dissolved with 20 mM PB (pH7.2, containing 300 mM NaCl, 8 M Urea and 20 mM Imidazole). After centrifugation at 15,000 rpm for 1 h, the supernatant was applied to a Ni2+-nitrilotriacetate (Ni-NTA) agarose column, washed with buffer A containing 50 mM imidazole, and eluted with buffer A containing 100 mM imidazole. Refolding was conducted under 4° C. The eluted proteins were first buffer exchanged to 1×PBS (pH7.4) containing 4 mM GSH, 0.4 mM GSSG, 0.4 M L-Arginine, 1 M Urea and 5% Glycerol then to PBS by dialysis. After the refolding, the protein solution was filtered by 0.22 μm filter and stored at −80° C.

### 4. Vaccination

Mice were randomized into 4 groups according to body weight and vaccinated three times as the table below:

| | | | | Dosing |
|---|---|---|---|---|
| Group | Immunization | Route | Number | Regimen |
| 1 | ROP-mSurvivin 100 ug + | S.C. | 10 | every 7 days |
| | 100 ul MPL | | | |
| 2 | ROP-mSurvivin 100 ug + | S.C. | 10 | every 7 days |
| | mSurvivin 100 ug + | | | |
| | 100 ul MPL | | | |
| 3 | PBS + 100 ul MPL | S.C. | 10 | every 7 days |
| 4 | PBS | S.C. | 10 | every 3 days |

### 5. ELISA

Purified mouse survivin or mouse ROP-Survivin (4 μg/ml) were coated onto flat-bottomed 96-well microtiter plates (Corning-Costar) in PBS overnight at 4° C. The wells were blocked with 5% BSA for 1 hour at room temperature. This followed by incubating with mice blood sera (1:10000 diluted in PBS) at room temperature for 1 hour. The binding was detected by using HRP-conjugated anti-mouse IgG secondary antibody. After washing, the plates were developed by adding 100 μl of TMB substrate solution. The reaction was stopped and the absorbance at 450 nm was measured using a spectrometer.

### Results

Purification of Mouse Survivin and Mouse ROP-Survivin

As can be seen in FIG. 7 the mouse survivin was purified and detectable using the mouse anti-His antibody. In the SDS-page, lane 1 shows a line indicative of the BSA control at the appropriate molecular weight (˜66 kDa), and lanes 2 and 3 show a band indicative of mouse survivin at the appropriate molecular weight (˜16 kDa). The Western blot shows that the His-tagged mouse survivin can be detected using a mouse anti-His antibody.

Likewise, FIG. 8 shows that the mouse ROP-survivin was purified and detectable using the mouse anti-His antibody. In the SDS-page (left hand side), lane 1 shows a line indicative of the BSA control at the appropriate molecular weight (˜66 kDa), and lanes 2 and 3 show a band indicative of the mouse ROP-survivin at the appropriate molecular weight (˜33 kDa). The Western blot shows that the His-tagged mouse ROP-survivin can be detected using a mouse anti-His antibody.

Detection of Antibodies Raised Against Mouse Survivin

FIG. 9 shows that administration of the mouse ROP-survivin alone and in combination with mouse survivin as described above produces much higher levels of antibody in mouse blood sera binding to mouse survivin coated plates. The ELISA results show that there was a significantly higher absorbance in both the mouse ROP-survivin and mouse ROP-survivin plus mouse survivin immunised groups compared to the MPL and PBS only groups, indicating that an immune response was raised against the ROP-survivin alone and in combination with mouse survivin (P<0.0001, one-way ANOVA with a post hoc test).

Remarkably, the co-administration of mouse ROP-survivin with mouse survivin produced a significantly higher absorbance in mouse blood sera from mice treated with the combination of the two compared to those treated with ROP-survivin alone (P<0.01, one-way ANOVA with a post hoc test). This indicates that the combination treatment is more effective at promoting an immune response than the ROP-survivin alone.

Detection of Antibodies Raised Against Mouse Survivin

FIG. 10 shows that administration of the mouse ROP-survivin alone and in combination with mouse survivin as described above produces much higher levels of antibody in mouse blood sera binding to mouse ROP-survivin coated plates. The ELISA results show that there was a significantly higher absorbance in both the mouse-ROP-survivin and mouse ROP-survivin plus mouse survivin immunised groups compared to the MPL and PBS only groups, indicating that an immune response was raised against the ROP-survivin alone and in combination with mouse survivin (P<0.0001, one-way ANOVA with a post hoc test).

As with the ELISA against mouse survivin coated plates, it would appear that remarkably the co-administration of mouse ROP-survivin with mouse survivin produced a significantly higher absorbance in mouse blood sera from mice treated with the combination of the two compared to those treated with ROP-survivin alone (P<0.001, one-way ANOVA with a post hoc test). This indicates that the combination treatment appears to provide an increased antibody response to both the mouse survivin and mouse ROP-survivin proteins.

### Example 3—Combining an HPV16 E7-ROP with the Native E7 Peptide

Materials and Methods

In order to validate the approach combining the native protein sequence with the polypeptide as described above, a mouse model using E7 peptide from HPV16 and a recombinant overlapping peptide (ROP) derived from HPV16 E7 with the ability to raise an immune response against HPV16 E7 is used. Though the below ROP is His-tagged, it will be understood that the ROP may be generated without said His-tag.

### 6. Sequences

His-tagged HPV16E7 protein
(SEQ ID NO: 48)
MHHHHHHGDTPTLHEYMLDLQPETTDLYCYEQLNDSSEEE
DEIDGPAGQAEPDRAHYNIVTFCCKCDSTLRLCVQSTHVD
IRTLEDLLMGTLGIVCPICSQKP
His-tagged HPV16E7-ROP
(SEQ ID NO: 49)
MHHHHHHGDTPTLHEYMLDLQPETTDLYCYEQLNDSSEEE
LRMKEQLNDSSEEEDEIDGPAGQAEPDRAHYNIVTFCCKL
RMKHYNIVTFCCKCDSTLRLCVQSTHVDIRTLEDLLMGLR
MKIRTLEDLLMGTLGIVCPICSQKP

### 7. Animals

Female C57BL/6 mice are purchased from Changzhou Kavins Experimental Animal Co. LTD. The animals are specific pathogen free and approximately 6-7 weeks old upon arrival. Upon receipt the animals are unpacked and placed in cages. A health inspection is performed on each animal to include evaluation of the coat, extremities and orifices. Each animal is also examined for any abnormal signs in posture or movement. The animals are housed in clear polycarbonate plastic cages (260 mm×160 mm×120 mm); 2-5 animals per cage. The bedding material is corn-cob bedding (irradiated, Shandong Goodway Biotechnology Co., Ltd., China) that is changed once a week. The room is supplied with HEPA filtered air at the rate of 15-25 air changes per hour. The temperature is maintained at 20-26° C. (68-79° F.). Illumination is fluorescent light for 12-hour light (08:00-20:00) and 12-hour dark. Animals have ad libitum access to rodent food (Shuck Beta Co., Ltd., China). Water, from the municipal water supply, is filtered by reverse osmosis or high-pressure sterilizer.

### 8. Expression/Purification

The expression of N-terminal His-tagged ROP-HPV16E7 or HPV16E7 protein is induced by 0.2 mM IPTG when the OD600 reached 0.5-0.8. The induction is performed at 15° C. for 16 hours.

For preparing bacterial lysates, the bacteria are suspended in 20 mM PB (pH7.2, containing 300 mM NaCl, 20 mM Imidazole, 1% Triton X-100, 1 mM DTT and 1 mM PMSF) and sonicated. Inclusion body (IB) is washed by 20 mM PB (pH7.2, containing 300 mM NaCl, 1% Triton X-100, 2 mM EDTA and 5 mM DTT). Finally, the cleaned IB is dissolved with 20 mM PB (pH7.2, containing 300 mM NaCl, 8 M Urea and 20 mM Imidazole). After centrifugation at 15,000 rpm for 1 h, the supernatant is applied to a Ni2+-nitrilotriacetate (Ni-NTA) agarose column, washed with buffer A containing 50 mM imidazole, and eluted with buffer A containing 100 mM imidazole. Refolding is conducted under 4° C. The eluted proteins are first buffer exchanged to 1×PBS (pH7.4) containing 4 mM GSH, 0.4 mM GSSG, 0.4 M L-Arginine, 1 M Urea and 5% Glycerol then to PBS by dialysis. After the refolding, the protein solution is filtered by 0.22 μm filter and stored at −80° C.

### 9. Vaccination

Mice are randomized into 4 groups according to body weight and vaccinated three times as the table below:

| | | | | Dosing |
|---|---|---|---|---|
| Group | Immunization | Route | Number | Regimen |
| 1 | ROP-HPV16E7 100 ug + | S.C. | 10 | every 7 days |
| | 100 ul MPL | | | |
| 2 | ROP-HPV16E7 100 ug + | S.C. | 10 | every 7 days |
| | HPV16E7 100 ug + | | | |
| | 100 ul MPL | | | |
| 3 | PBS + 100 ul MPL | S.C. | 10 | every 7 days |
| 4 | PBS | S.C. | 10 | every 3 days |

### 10. ELISA

Purified HPV16E7 or ROP-HPV16E7 (4 μg/ml) are coated onto flat-bottomed 96-well microtiter plates (Corning-Costar) in PBS overnight at 4° C. The wells are blocked with 5% BSA for 1 hour at room temperature. This is followed by incubating with mice blood sera (1:10000 diluted in PBS) at room temperature for 1 hour. The binding is detected by using HRP-conjugated anti-mouse IgG secondary antibody. After washing, the plates are developed by adding 100 μl of TMB substrate solution. The reaction is stopped and the absorbance at 450 nm is measured using a spectrometer.

Results

The results will show that the HPV16E7 and ROP-HPV16E7 are successfully purified and can be specifically detected using anti-His antibody by SDS-page and Western blot, with BSA acting as a control.

In addition, the results will show that the combination of ROP-HPV16E7 and HPV16E7 is more effective at raising an antibody response detected in mouse blood sera using an ELISA as outlined above than that raised from mice immunised using ROP-HPV16E7 alone.

### Example 4—ELISA Measurement of Antibody Generation Against SARS-CoV-2 Vaccine Formulations

Immunisation

10 Mice per immunisation group (below) were immunised by subcutaneous injection on day 0, day 14, day 21, and day 28 as follows:
Group 1—ROP-COVS 100 ug
Group 2—RBM 50 μg
Group 3—ROP 50 μg+RBM 50 μg
Group 4—PBS (negative control)

On day 35, the mice were bled in preparation for ELISA testing of serum to determine antibody generation in response to the above vaccination protocol.

The RBM used for immunisation in this assay corresponds to SEQ ID NO: 51. The ROP used for immunisation in this assay corresponds to SEQ ID NO: 44

ELISA Measurement of Antibodies

A 96-well plate was coated with 100 μl per well of a 2 μg/ml of RBD (SEQ. ID NO: 50) solution in PBS overnight at 4° C. The plate was then washed with PBS before being incubated with 200 μl per well of a 2.5% (w/v) solution of BSA at 37° C. for 1 hour. The plate was again washed with PBS before 100 μl of mouse serum diluted at different serum titres was added to each well and incubated at 37° C. for 1 h. The plate was washed prior to the addition of goat anti-mouse-HRP antibody at 1:20000 in PBS, 50 μl per well, incubated at room temperature for 30 minutes.

The plate was washed prior to the addition of 100 μl of TMB colour developing solution, before being incubated for 5-10 minutes following the manufacturer's instructions. 50 μl of stop solution was added to each well prior to the measurement of absorbance at OD450 nm on a spectrometer.

FIG. 11 shows the results of the ELISA. The graph shows the absorbance at 450 nm for the different sera dilutions for each immunisation group. It is clear that there was more absorbance in the group immunised with both the RBM and ROP compared to either the RBM or ROP alone. There was little absorbance in the negative control group as expected. Synergy between the ROP and RBM is shown by virtue of the fact that 50 μg RBM and 50 μg ROP produced greater absorbance than 50 μg RBM alone or 100 μg ROP alone, with the difference being statistically significant for 4 of the dilution titres (p<0.05). Additionally, the combination group is resistant to dilution, with significantly greater absorbance than the other three groups at dilutions of 1:102400, 1:409600, and 1:1638400. This shows either that the antibody response produced by the combination approach involves higher affinity antibodies or a greater abundance thereof. Synergy is demonstrated as the response of a lower dose (50 μg) of ROP combined with the 50 μg of RBM produces a greater response than 100 μg of ROP, and this cannot be explained by mere additive effect.

### Example 5—Restimulation of Splenocytes from Mice Immunised with Survivin Vaccine Formulations

Splenocytes were isolated according to standard protocols from mice immunised subcutaneously, weekly for a period of 3 weeks according to the following table:

| Group | No. | Test Articles and Doses |
|---|---|---|
| ROP + | 10 | 100 ug mROP-survivin + 100 ug mSurvivin + |
| survivin | | MPL |
| ROP vaccine | 10 | 100 ug mROP-survivin + MPL |
| Survivin | 10 | 100 ug mSurvivin + MPL |
| vaccine | | |
| PBS | 10 | PBS |

The splenocytes (2×10⁵ cells per well) from each group were restimulated with 5 μg/well of either ROP, survivin, or PHA in PBS in an ELISPOT assay. The negative control was an addition of the same PBS buffer but without a stimulant. The results are shown in FIG. 12.

The results show that the splenocytes from the ROP-survivin plus survivin group produce a greater response upon restimulation with ROP-survivin (p<0.001) and Survivin (p<0.01) than the survivin only vaccinated group. This indicates that the ROP-survivin plus survivin vaccinated mice produce a greater immune response when challenged with a survivin-based antigen compared to mice immunised with either vaccine alone. ROP combined with survivin produces a strong T-cell response, which in part explains the synergetic effect of the combination approach over the native protein alone or the ROP alone. The ROP T cell response amplifies the antibody response to the native protein in the combined approach to produce effects over and above that of either antigen alone.

### REFERENCES

Bartsch S M, O'Shea K J, Ferguson M C, et al. Vaccine Efficacy Needed for a COVID-19 Coronavirus Vaccine to Prevent or Stop an Epidemic as the Sole Intervention. Am J Prev Med. 2020; 59(4):493-503. doi:10.1016/j.amepre.2020.06.011
Cai, L., Zhang, J., Zhu, R., Shi, W., Xia, X., Edwards, M., Finch, W., Coombs, A., Gao, J., Chen, K., Owen, S., Jiang, S., & Lu, W. (2017). Protective cellular immunity generated by cross-presenting recombinant overlapping peptide proteins. Oncotarget, 8(44), 76516-76524. https://doi.org/10.18632/oncotarget.20407
Hollingsworth, R. E., Jansen, K. Turning the corner on therapeutic cancer vaccines. npj Vaccines 4, 7 (2019). https://doi.org/10.1038/s41541-019-0103.y
Zhang H, Hong H, Li D, Ma S, Di Y, Stoten A, Haig N, Di Gleria K, Yu Z, Xu X N, McMichael A, Jiang S. (2009) Comparing pooled peptides with intact protein for accessing cross-presentation pathways for protective CD8+ and CD4+ T cells. The Journal of Biological Chemistry. 284(14) 9184-9191. https://doi.org/10.1074/jbc.m809456200

1. A formulation, comprising:
(i) a polypeptide comprising two or more peptide fragments and one or more protease cleavage site sequences located between each of the two or more peptide fragments, wherein a first peptide fragment comprises a first sequence derived from a native protein sequence and wherein a second peptide fragment comprises a second sequence derived from the native protein sequence; and
(ii) the native protein sequence or a portion thereof.

2. The formulation of claim 1, comprising one or more overlapping sequences between 2 and 31 amino acids in length, optionally wherein the one or more overlapping sequences are 8 to 31 amino acids in length.

3. The formulation of claim 1, wherein the one or more protease cleavage site sequences is an exogenous protease cleavage site.

4. The formulation of claim 4, wherein the protease cleavage site sequence is a cathepsin cleavage sequence selected from cathepsin S and LRMK cleavage sequence.

5. The formulation of claim 1, further comprising a pharmaceutically acceptable carrier.

6. The formulation of claim 1, further comprising an adjuvant selected from Monophosphate Lipid A (MPL), montanide, alum-based adjuvants, oil-in-water adjuvants, and water-in-oil adjuvants.

7. The formulation of claim 1, wherein the polypeptide is in a concentration between 10 to 10000 μg·kg⁻¹ and the native protein sequence or portion thereof is in a concentration between 10 to 10000 μg·kg⁻¹.

8. The formulation of claim 1, wherein the native protein sequence is the S protein of a coronavirus.

9. The formulation of claim 8, wherein the coronavirus is a betacoronavirus, a severe acute respiratory syndrome-related coronavirus, or SARS-CoV-2.

10. The formulation of claim 1, wherein at least two of the two or more peptide fragments of the polypeptide comprise sequences derived from the S1 and/or S2 subunit of the S protein and/or wherein the portion of the native protein sequence comprises sequences derived from the S1 and/or S2 subunit of the S protein.

11. The formulation of claim 10, wherein at least one of the two or more peptide fragments comprises a sequence derived from the receptor binding domain (RBD), optionally the receptor binding motif (RBM) of the S1 subunit and/or wherein the portion of the native protein sequence comprises the receptor binding domain (RBD), optionally the receptor binding motif (RBM) of the S1 subunit.

12. The formulation of claim 10, wherein at least one of the two or more peptide fragments comprises a sequence derived from the HR2 and/or HR1 domain of the S2 subunit and/or wherein the portion of the native protein sequence comprises the HR2 and/or HR1 domain of the S2 subunit.

13. The formulation of claim 1, wherein the native protein sequence is survivin, chosen from any one of the following survivin isoforms: Isoform 1, Isoform 2, Isoform 3, Isoform 4, Isoform 5, Isoform 6, and Isoform 7.

14. The formulation of claim 13, wherein at least one of the two or more peptide fragments comprises a sequence with at least 90% identity to a sequence selected from:
MGAPTLPPAWQPFLKDHRISTFKNWPFLEG,
DHRISTFKNWPFLEGCACTPERMAEAGFIH
ACTPERMAEAGFIHCPTENEPDLAQCFF,
PTENEPDLAQCFFCFKELEGWEPDDDPIE,
FKELEGWEPDDDPIEEHKKHSSGCAFLSVK,
EHKKHSSGCAFLSVKKQFEELTLGEFLK,
QFEELTLGEFLKLDRERAKNKIAKETNNK,
RERAKNKIAKETNNKKKEFEETAEKVRRAI,
and/or
KEFEETAEKVRRAIEQLAAMD,
and
wherein the polypeptide elicits an immune response or is immunostimulatory.

15. The formulation of claim 13, wherein the two or more peptide fragments comprise a sequence with at least 90% identity to:
PTENEPDLAQCFFCFKELEGWEPDDDPIE
and/or
FKELEGWEPDDDPIEEHKKHSSGCAFLSVK,
and
wherein the polypeptide elicits an immune response, optionally a T-cell response.

16. The formulation of claim 1, wherein the native protein sequence is an E6 or E7 protein of a Human papillomavirus (HPV).

17. The formulation of claim 16, wherein the native protein sequence is:
MHQKRTAMFQ DPQERPRKLP QLCTELQTTI HDIILECVYC
KQQLLRREVY DFAFRDLCIV YRDGNPYAVC DKCLKFYSKI
SEYRHYCYSL YGTTLEQQYN KPLCDLLIRC INCQKPLCPE
EKQRHLDKKQ RFHNIRGRWT GRCMSCCRSS RTRRETQL;
or
MHGDTPTLHE YMLDLQPETT DLYCYEQLND SSEEEDEIDG
PAGQAEPDRA HYNIVTFCCK CDSTLRLCVQ STHVDIRTLE
DLLMGTLGIV CPICSQKP.

18. The formulation of claim 16, wherein at least one of the two or more peptide fragments comprises a sequence with at least 90% identity to a sequence selected from:
MHGDTPTLHEYMLDLQPETTDLYCYEQLNDSSEEE,
EQLNDSSEEEDEIDGPAGQAEPDRAHYNIVTFCCK,
HYNIVTFCCKCDSTLRLCVQSTHVDIRTLEDLLMG,
and/or
IRTLEDLLMGTLGIVCPICSQKP.

19. A formulation comprising: one or more polynucleotides encoding the native protein sequence or portion thereof and/or one or more polynucleotides encoding the polypeptide of any one of claim 1.

20. A method for the immunization and/or treatment of a subject comprising: administering, to the subject, the formulation of claim 1.

21. A method of manufacturing a vaccine comprising: expressing one or more polynucleotides encoding the native protein sequence or portion thereof and the polypeptide according to claim 1, in one or more cells in vitro, and purifying the native protein sequence or portion thereof and the polypeptide.

22. A method for the immunization and/or treatment of a subject comprising:
(a) administering the native protein sequence or portion thereof of according to claim 1, or one or more polynucleotides encoding the native protein sequence or portion thereof, and
(b) administering the polypeptide according to claim 1, or one or more polynucleotides encoding the polypeptide.

23. The method according to claim 22, wherein the native protein sequence or portion thereof, or one or more polynucleotides encoding the native protein sequence or portion thereof are administered simultaneously, sequentially, or separately to the polypeptide or one or more polynucleotides encoding the polypeptide.
